(19) United States
(12) Patent Application Publication
(10) Pub. No.: US 2014/0171790 A1
(43) Pub. Date: Jun. 19, 2014

(54) METHOD AND APPARATUS FOR THREAD TRANSECTION OF A BODY TISSUE

(71) Applicant: Joseph Guo, Monterey Park, CA (US)

(72) Inventor: Joseph Guo, Monterey Park, CA (US)

(21) Appl. No.: 14/186,931

(22) Filed: Feb. 21, 2014

Related U.S. Application Data
(63) Continuation-in-part of application No. 13/870,291, filed on Apr. 25, 2013.
Application No. 13/870,291 is a continuation-in-part of application No. 13/460,246, filed on Apr. 30, 2012.

(51) Int. Cl. A61B 17/32; A61B 19/02; A61B 8/08

(52) U.S. Cl. CPC A61B 17/320036; A61B 8/0841; A61B 17/32002; A61B 19/0264
USPC 600/424; 606/171; 606/170

(57) ABSTRACT

A flexible thread-like cutting element achieves a smooth and sharp cutting action by utilizing a power tool or manual tool capable of developing a reciprocating motion that alternatively reciprocates the ends of the cutting element. The tool may include a mechanism for converting rotational motion into reciprocating motion. The reciprocating motion is transferred to the cutting element to develop the cutting action of the device. In another aspect, the power tool or manual can be encased in a disposable, sterile protective bag to simplify sterilization.

### CROSS-REFERENCES TO RELATED APPLICATIONS

This application is a continuation-in-part of U.S. Ser. No. 13/870,291, filed on Apr. 25, 2013, incorporated by reference herein in its entirety, which is a continuation-in-part of U.S. Ser. No. 13/460,246, filed on Apr. 30, 2012, incorporated by reference herein in its entirety.

The present invention is generally directed to a surgical method for transecting a soft body tissue, such as a ligament or tendon, and to an apparatus for performing such method. More particularly, the present invention provides a hand-held power tool or manual tool which can utilize various mechanisms for developing alternating releasing-pulling actions that can be transmitted to the ends of a thin and flexible thread-like cutting element to allow the cutting element to transect, by minimally invasive means, soft tissue such as the transverse carpal ligament that is commonly released as a treatment for carpal tunnel syndrome. The present invention is also directed to a protective sterile bag which encases the power tool or manual tool to simplify sterilization.

### BACKGROUND OF THE INVENTION

Many people suffer from injury to the soft tissues of the wrist and carpal tunnel, often caused by frequent, sustained repetitive motion involving the hands. Repetitive activities which require the same or similar hand/wrist action can result in injuries which have been collectively referred to as Cumulative Repetitive Stress Syndrome or Repetitive Strain Injury. The most familiar and common of such wrist injuries is known as carpal tunnel syndrome which produces pain, discomfort, nerve conduction disturbances, and impairment of function of the hand and sometimes the arm as well. The most common symptoms of this condition include intermittent pain and numbness of the hand.

Carpal tunnel syndrome occurs when the median nerve which runs from the forearm into the hand, becomes pressed or squeezed at the wrist. The median nerve provides feeling in one's thumb and along with index, middle and ring ringers. The median nerve controls sensations to the palmar side of the thumb and these fingers as well as impulses to some muscles in the hand which allow the fingers and thumb to move. The median nerve receives blood, oxygen and nutrients through a microvascular system which is present in the connective tissue surrounding the nerve fiber. Increased pressure on the nerve fiber can constrict these microvessels and will reduce the blood flow to the median nerve. Any prolonged deprivation of oxygen and nutrients can result in severe nerve damage.

The median nerve passes through the carpal tunnel, a canal in the wrist surrounded by the carpal bones on three sides and a fibrous sheath called the transverse carpal ligament on the fourth side. In addition to the median nerve, the nine flexor tendons in the hand pass through this canal. When compressed, the median nerve will cause pain, weakness or numbness in the hand and wrist which may also radiate up along he arm. The median nerve can be compressed by a decrease in the size of the carpal canal itself or an increase in the size of its contents (i.e. such as the swelling of the flexor tendons and of the lubrication tissue surrounding these flexor tendons), or both. For example, conditions that irritate or inflame the tendons can cause them to swell. The thickening of irritated tendons or swelling of other tissue within the canal narrows the carpal canal, causing the median nerve to be compressed. The cross-sectional area of the tunnel also changes when the hand and wrist changes positions. Wrist flexion or extension can decrease the cross-sectional area, thus increasing the pressure exerted on the median nerve. Flexion also causes the flexor tendons to somewhat rearrange which can also compress the median nerve. For example, simple bending of the wrist at a 90 degree angle will decrease the size of the carpal canal. Without treatment, carpal tunnel syndrome can lead to chronic neural muscular disorders of the hand and sometimes the arm.

Treatment for carpal tunnel syndrome includes a variety of non-surgical as well as surgical procedures, wherein carpal tunnel release is one of the most common surgical procedures that is performed. Such surgery involves the severing of the transverse carpal ligament to relieve the pressure on the median nerve and is commonly performed via either open or endoscopic methods. In open methods, the skin lying over the carpal tunnel is incised after which the transverse carpal ligament is transected under direct vision. The skin is then reapproximated with sutures. Endoscopic methods require incision of the skin in one or more locations to allow for the insertion of an endoscope along with various tools that are needed to transect the ligament. Such tools typically include a combination of a specially configured scalpel and guide instrument. The insertion of such tools into proper position below, above or both below and above the target ligament further requires the formation of one or more pathways in the hand with attendant trauma to the surrounding tissue and the potential for nerve damage as well as a more protracted post-surgical healing process. Additionally, the use of a scalpel typically requires multiple passes thereof in order to complete a transection which causes a complex pattern of cuts to be imparted onto the severed ligament surfaces.

Less invasive techniques have been proposed including for example the use of flexible saw elements that are introduced into the hand and positioned adjacent to or wrapped about a portion of the target ligament after which the saw element is reciprocated to cut the tissue. A substantial disadvantage of a cut that is made by a saw-like instrument as opposed to a knife-like instrument is inherent in the fact that a kerf is created. The material that is removed from the kerf is either deposited in and around the surgical site or additional steps must be taken to retrieve such material. Additionally, the cut surfaces that are created by a saw tend to be relatively rough and abraded with microtrauma on the cutting surface that may increase inflammatory response (edema, erythema, heat and pain), could result in local tissue adhesions and scarring which can delay or complicate the healing process.

Alternatively, techniques have been proposed wherein a taut wire, string or filament is used to cut a ligament. The cut is achieved either by the tautening of the cutting element or alternatively, by reciprocating the taut element. Disadvantages associated with such an approach are inherent in the less than optimal geometry by which a taut wire can be brought to bear on the target ligament and by the invasiveness of the tightening apparatus.

A new method and apparatus was needed with which tissue such as a ligament or a tendon can be percutaneously accessed and transected so as to cause a very minimal amount of disruption to the surrounding tissue and by which a smooth, kerf-less cut is achieved. Methods and apparatus to address such tissue transection have been addressed in my co-pending U.S. patent application Ser. Nos. 13/870,291 and 13/460,246. What is further needed is a power tool or a manual tool for reciprocating the ends of the cutting element used to create the smooth, kerf-less cut in such a tissue transection. Moreover, it would certainly be beneficial if such a power tool or a manual tool could be kept satisfactorily sterilized to allow the tool to be used in several tissue transection procedures without the need to repeatedly re-sterilize the power tool. The present invention satisfies these and other needs.

### SUMMARY OF THE INVENTION

The present invention provides for the minimally-invasive transection of soft tissue, such as, a ligament utilizing a thin and flexible thread-like cutting element that achieves a smooth and sharp cutting action by utilizing a power tool capable of developing a reciprocating motion that alternatively reciprocates the ends of the cutting element. The method and apparatus obviate the need for large incisions, while minimizing disruption of the tissue surrounding the target tissue. The present invention also enables a smooth kerf-less cut of the target tissue to be achieved, usually requires no suturing and can be easily and quickly performed in a clinic setting. In another aspect, the power tool can be encased in a disposable, sterile protective bag which allows the power tool to be used a number of times before the power tool itself has to be sterilized. After the procedure is completed, the previously used bag can be quickly removed from the power tool and disposed of properly and a new protective bag can be placed over the power tool for new procedure.

More particularly, the invention provides for the introduction of a thin and flexible thread-like cutting element into the body and its routing about the target soft tissue, such as a ligament or tendon. Subsequent manipulation of the protruding ends of the smooth cutting element serves to transect the ligament by a smooth kerf-less cut. A routing tool component of the invention enables the cutting element to be easily and quickly introduced and routed into position about the target tissue (ligament or tendon) with minimal disruption or trauma to the surrounding tissue. For example, in one aspect, the routing tool component may take the form of a hollow introducer needle or a specially configured hooked retrieval needle comprising a thin, rigid and elongated needle-like element having near its distal end a hook-like feature formed therein that is dimensioned to engage the cutting element and configured to maintain engagement therewith when being pulled proximally.

The very small cross-section of the routing tool, whether it takes the form of the hollow introducer needle or the hooked retrieval needle, and of the cutting element, as well as minimally invasive method by which such hardware is introduced and positioned within the hand greatly reduces the risk of injury to the median nerve as well as to the smaller nerves that branch out therefrom. Additionally, the fact that the cutting element is positioned via only two tiny punctures and that the transection is performed via only one of those punctures, recovery time is minimal and scaring is essentially negligible. Reciprocation of the thread-like cutting element may preferably be achieved with the use of a power tool.

In one aspect of the present invention, the reciprocation of the thread-like cutting element may be achieved by a power tool which develops an alternating releasing-pulling action on the ends of the cutting element. In one aspect, the power tool includes a drive motor that rotates a shaft and a mechanism for converting the rotational motion of the shaft into a reciprocating motion. The reciprocating motion that is developed by the conversion mechanism can then be used to alternatively reciprocate the ends of the cutting element. In another aspect of the present invention, a system for transecting soft tissue within a body would include the flexible thread-like cutting element having a first end and a second end, a routing tool configured to facilitate its extension into the body adjacent to the soft tissue and routing the thread-like cutting element about the soft tissue such that both ends of the cutting element extend from a single access port in the body and the power tool which develops the reciprocating motion that alternatively reciprocate the ends of the cutting element.

The power tool may include a housing or body including a handle portion for grasping the body, a drive motor that rotates a shaft housed within the body and a power source for powering the drive motor. A power switch is used to actuate and control the speed of the drive motor. The power tool can have variable speeds to allow the surgeon to develop the desired alternating releasing-pulling action needed to create a suitable cutting speed for the cutting element. In one aspect of the invention, an actuating mechanism is coupled to the drive motor for developing the alternating releasing-pulling action on the ends of the cutting element. In one particular aspect, the actuating mechanism includes a pair of rotating discs with a crankpin located on each of the rotating discs. Each crankpin has a locking pin attached to it. The cutting element is attached to each of the locking pins so that as the rotating discs move each crankpin, the alternating releasing-pulling action is transferred to the cutting element. In one particular aspect, the actuating mechanism which is driven by the motor of the power tool includes a cylinder having a track formed on the outer surface of the cylinder. The cylinder is rotatable by the drive means of the power tool. A first track follower and a second track follower are placed within the track of the cylinder and move in a reciprocating manner as the cylinder rotates. In another aspect, the actuating mechanism includes a first cam, a first follower in contact with the first cam and movable in a reciprocating manner, a second cam and a second follower in contact with the second cam and movable in a reciprocating manner in opposite direction of the first follower. In another aspect, the actuating mechanism includes a crankpin, a crank connecting rod having a first end and a second end, the first end of crank connecting rod being engaged with the crankpin and a rocking arm engaged with the second end of crank connecting rod and movable in a rocking manner. An alternative embodiment of the actuating mechanism includes a cam and a rocking arm with a follower having a fork-like structure on one end, the rocking arm being in contact with the cam by the follower and movable in a rocking manner. The function of the power tool can also be achieved by a manually operated hand tool powered by the mechanism of a hand-turning crank and designed with the actuating mechanisms described above.

The physical characteristics of the cutting element are selected to facilitate a kerf-less cut through the soft tissue. The small diameter and high tensile strength of the cutting element provides for the transection of the ligament or tendon by the manipulation of the ends of the cutting element. Unequal forces can alternatingly be applied to the two ends of the cutting element to induce a reciprocating cutting action. Alternatively, one end can be pulled with greater force than the other element so as to pull the cutting element in a single direction as it cuts through the ligament or tendon. As a further alternative, both ends can be pulled simultaneously with equal force to simply pull the cutting element through the ligament or tendon. The substantially smooth, none abrasive surface of the cutting element causes a knife-like cut to be achieved without the formation of a kerf and thus without an attendant deposition of detached material in and about the surgical site.

In yet another aspect of the present invention, a disposable isolative system for encasing a power tool or a manual tool capable of achieving alternating releasing-pulling action to the cutting element which comprises a thin wall protective bag made from a flexible and soft material which is able to be sterilized and impermeable to germs and/or bacteria. The protective bag includes an opening for receiving the tool, a locking component associated with the opening for sealing the opening and a mechanism of transferring the alternative releasing-pulling actions of the tool to the outside of the bag and to the cutting element. The protective bag allows the tool to be used numerous times before the power tool has to be re-sterilized. The protective bag is disposable after each use so that a new protective bag could be easily placed on the tool after each patient use. The bag can be made from a soft plastic material or plastic-like material which is impermeable to germs and/or bacteria. The protective bag would be made with a thin wall so that the bag is soft and flexible. The protective bag thus forms a protective barrier to germs and bacteria while allowing the user to properly hold the tool and actuate the power switch. The protective bag can be formed to substantially match the profile of the tool allowing the user to properly grasp the handle portion and actuate the power switch of the tool or operate the hand-turning crank from outside the bag. The protective bag includes mechanisms which allow the alternating releasing-pulling action developed by the tool within the bag to be transferred outside of the bag. As such, the tool remains encased within the protective bag while the moving parts of the tool are transferred to components outside of the bag. The cutting element can be easily attached to these outside elements in order to develop the alternating releasing-pulling action on the cutting element. These and other advantages of the present invention will become apparent from the following detailed description of preferred embodiments which, taken in conjunction with the drawings illustrate by way of example the principles of the invention.

### BRIEF DESCRIPTION OF THE DRAWINGS

FIG. 1 is a cross-sectional view of the carpal tunnel area of the hand.

FIG. 2 is a perspective view of a preferred embodiment of the routing tool component of the present invention in the form of a hooked retrieval needle.

FIG. 3 is a perspective view of a preferred embodiment of the cutting element of the present invention.

FIGS. 4A-H are cross-sectional views of the hand with a revealed transverse carpal ligament illustrating a preferred sequence of steps for practicing the method of the present invention using a hooked retrieval needle as the routing tool component.

FIG. 5 is a perspective view of another preferred embodiment of the routing tool component of the present invention in the form of a hollow introducer needle.

FIG. 6 is a perspective view of another preferred embodiment of the cutting element of the present invention.

FIGS. 7A-J are cross-sectional views of the hand with a revealed transverse carpal ligament illustrating a preferred sequence of steps for practicing the method of the present invention using a hooked retrieval needle as the routing tool component.

FIG. 8 is a perspective view of an embodiment of a power tool for providing an alternating releasing-pulling action on the cutting element (not shown) once the cutting element has been positioned about the target body tissue.

FIG. 9 illustrates the power tool of FIG. 8 being used to provide the alternating releasing-pulling action on the cutting element once the cutting element has been placed about a ligament in the patient's wrist.

FIG. 10 is a schematic drawing showing an embodiment of a mechanism using two crankpins that can be used in conjunction with the power tool for achieving the alternating releasing-pulling action to move the flexible cutting element.

FIG. 11 is a schematic drawing showing an embodiment of a mechanism using a cylinder track and two followers that can be used with the power tool for achieving the alternating releasing-pulling action needed to move the flexible cutting element.

FIG. 12 is a schematic drawing of an embodiment of a mechanism using two cams and two followers that can be used in conjunction with the power tool for achieving the alternating releasing-pulling action needed to move the flexible cutting element.

FIG. 13 is a schematic drawing of an embodiment of a mechanism using a four-bar linkage that can be used in conjunction with the power tool for achieving the alternating releasing-pulling action needed to move the flexible cutting element.

FIG. 14 is a schematic drawing of an embodiment of a mechanism using a cam and rocker with a forked follower that can be used in conjunction with the power tool for achieving the alternating releasing-pulling action needed to move the flexible cutting element.

FIG. 15 is a side elevational view of a disposable, isolative system for encasing a power tool made in accordance with the present invention.

FIG. 16 is a cross sectional view of the disposable, thin wall bag of the isolative system of FIG. 15 taken along line 16-16.

FIG. 17 is a side view partially in cross section of the mechanism for transferring the alternating releasing-pulling action of the power tool to the outside of the thin wall bag disclosed in FIG. 16.

FIG. 18 is side view partially in cross section of another embodiment of a mechanism for transferring the alternating releasing-pulling action of the power tool to the outside of the thin wall bag.

FIG. 19 is side view partially in cross section of another embodiment of a mechanism for transferring the alternating releasing-pulling action of the power tool to the outside of the thin wall bag.

FIG. 20 is side view partially in cross section of a hollow needle and inner needle having a distal tip portion which is bendable to a desired direction.

FIG. 21A is side view partially in cross section of a hollow needle and inner needle made from a Nitinol alloy having a distal tip portion which movable from a substantially straight position to a pre-formed curved position, as is shown in FIG. 21C, when the inner needle is subjected to body heat.

FIG. 21B is side view partially in cross section of the hollow needle and inner needle of FIG. 21A as it gradually moves from the substantially straight position to the pre-formed curved position as shown in FIG. 21C.

FIG. 21C is side view partially in cross section of the hollow needle and inner needle of FIGS. 21A and 21B showing the distal tip portion in its final pre-formed curved position.

### DETAILED DESCRIPTION OF THE PREFERRED EMBODIMENTS

The present invention provides for the minimally invasive transection of tissue such as, but not limited to, ligaments and tendons, and obviates the need for scalpels, saws or endoscopes. The invention is especially applicable for the transection of ligaments and most particularly, for the release of the transverse carpal ligament in the treatment of carpal tunnel syndrome.

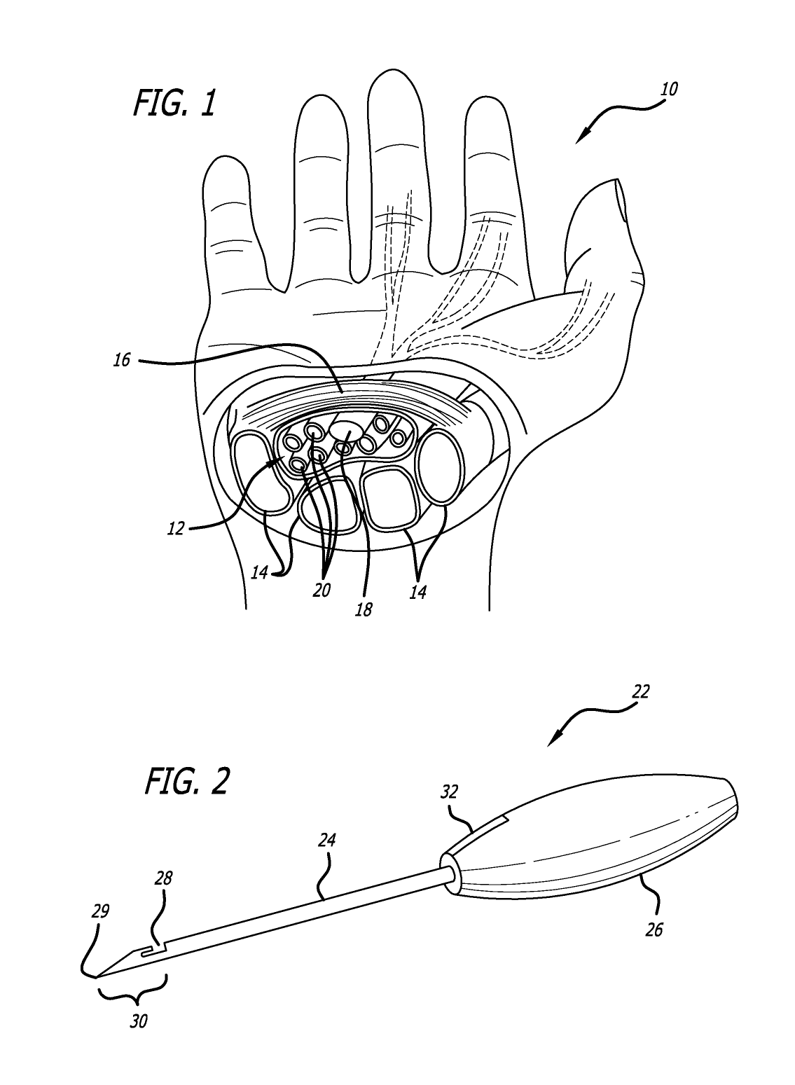

FIG. 1 is a cross-sectional view of the carpal tunnel area of the hand 10. The carpal tunnel 12 is the area of the wrist and palm of the hand 10 formed by a U-shaped cluster of bones 14 that form a hard floor and two walls of the tunnel. The roof of the tunnel is formed by the transverse carpal ligament 16 which attaches to the wrist bones. Within the confines of the tunnel is the median nerve 18 and the flexor tendons 20 of the thumb and fingers. Carpal tunnel syndrome is caused by a compression of the median nerve by either a decrease in the size of the tunnel or an increase in the size of its contents. Such pressure may be relieved by a release of the ligament such as by a transection thereof.

FIG. 2 is perspective view of a preferred embodiment of the routing tool of the present invention wherein such tool takes the form of a hooked retrieval needle 22. The tool generally includes a thin, rigid and elongated distal section 24 and a handle 26 at its proximal end. The distal section has hooking element 28 disposed near its distal end 30. The hooking element is preferably defined by a void formed within the outer diameter of the elongated distal section of the retrieval tool so as to present a substantially smooth outer surface and thereby minimize the potential for trauma as the tool is extended into or retracted from tissue. The distal end may have a sharp tip 29 as is shown in the illustrated embodiment. Alternatively, the tip may have a more blunted configuration. The hooking element is spaced slightly back (reference numeral 30) from the distal end. A marking 32 on the handle may be included demarking the rotational position of the hook-like feature near the tool's distal end. The length of the distal section is selected to be greater than the width of the transverse carpal ligament. Its diameter is selected to be no greater than about 1 mm.

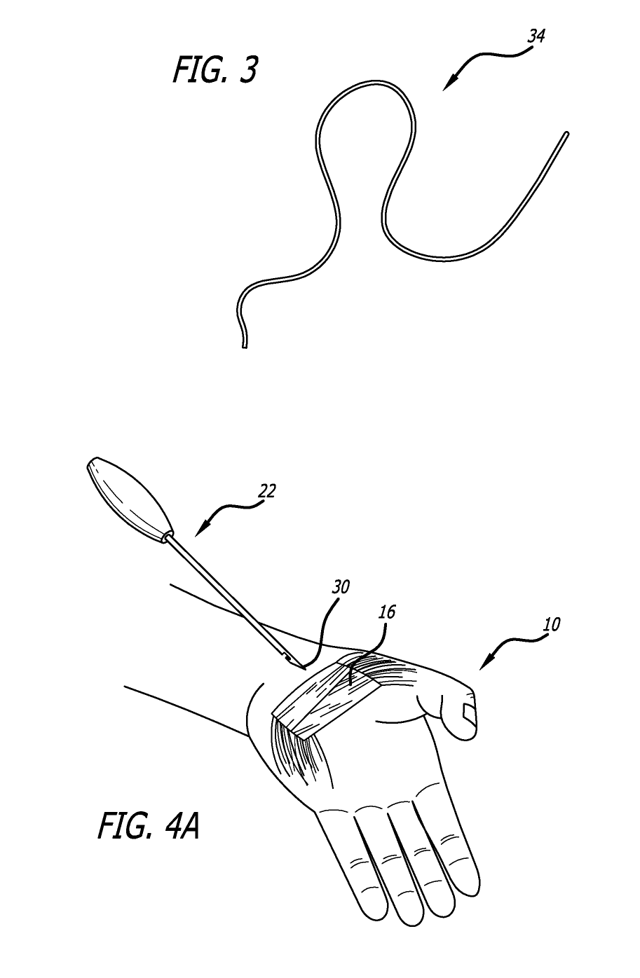

FIG. 3 is a perspective view of the cutting element 34 of the present invention The cutting element has a flexible, small diameter, thread-like structure with a high tensile strength and a smooth and soft surface. The cutting element may comprise a monofilament or a plurality of braided, twisted or otherwise joined fibers or strands wherein each strand has a smooth surface so as to present a relatively smooth, none-abrasive surface. Its physical characteristics include a bend radius of less than half the thickness of the ligament and preferably a zero bend radius, a diameter of less than about 1.0 mm, and a breaking strength of over 2 lbs. The cutting element may comprise fiber or yarn formed of cotton, silk, glass fiber, carbon fiber, various plastic fibers or metal. More particularly, textile fiber, synthetic fiber, mineral fiber, polymer fiber, microfibers may be used. A handle may be disposed near its proximal end to enable the tool to be grasped and manipulated. At least one end of the cutting element may include a stiffened section to facilitate the introduction into and the extension through a hollow introducer needle. The stiffened section may be formed by covering the section with relatively stiff tubing, by subjecting a synthetic fiber to heat, by the infusion of for example a resin, by coating a glue or by the attachment of for example a suture needle. The stiffened section preferably has a diameter less than the inner diameter of the introducer needle.

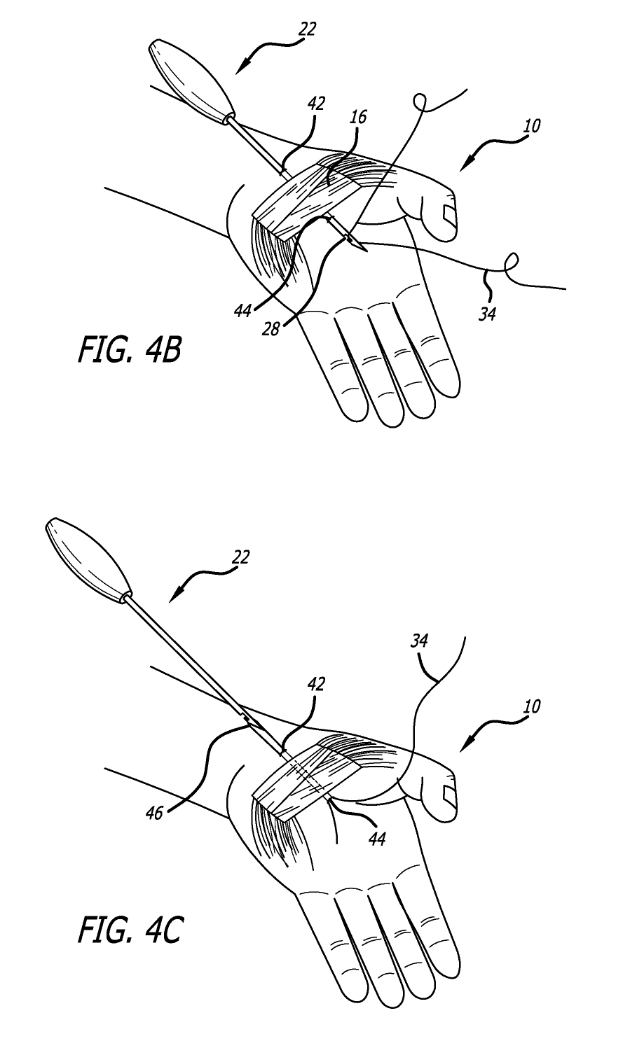

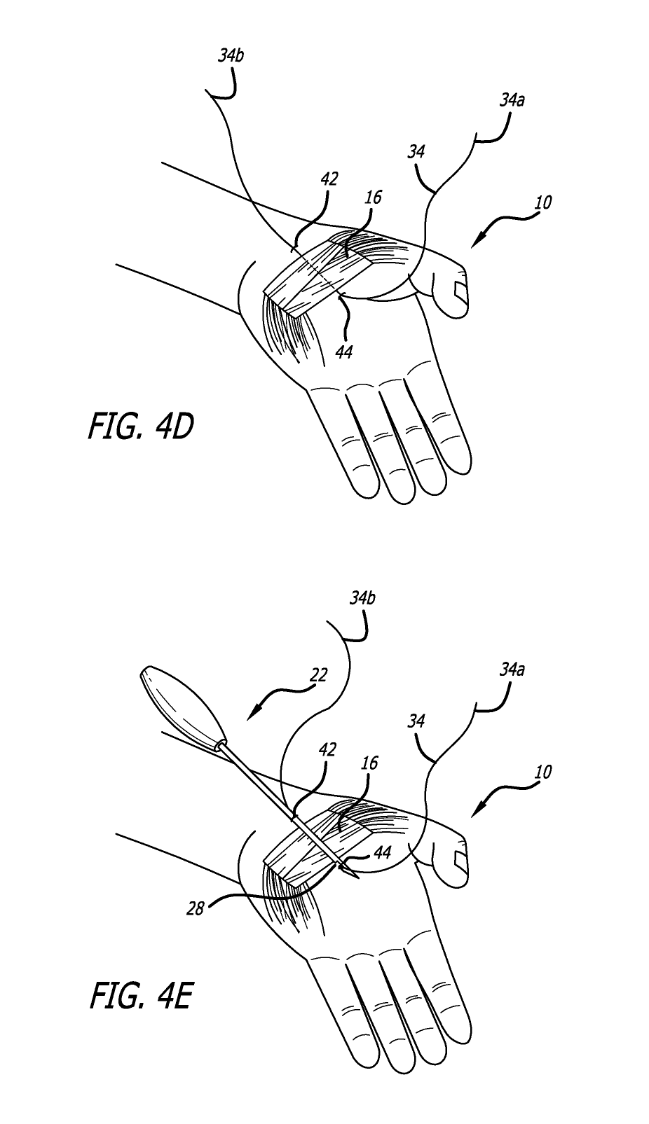

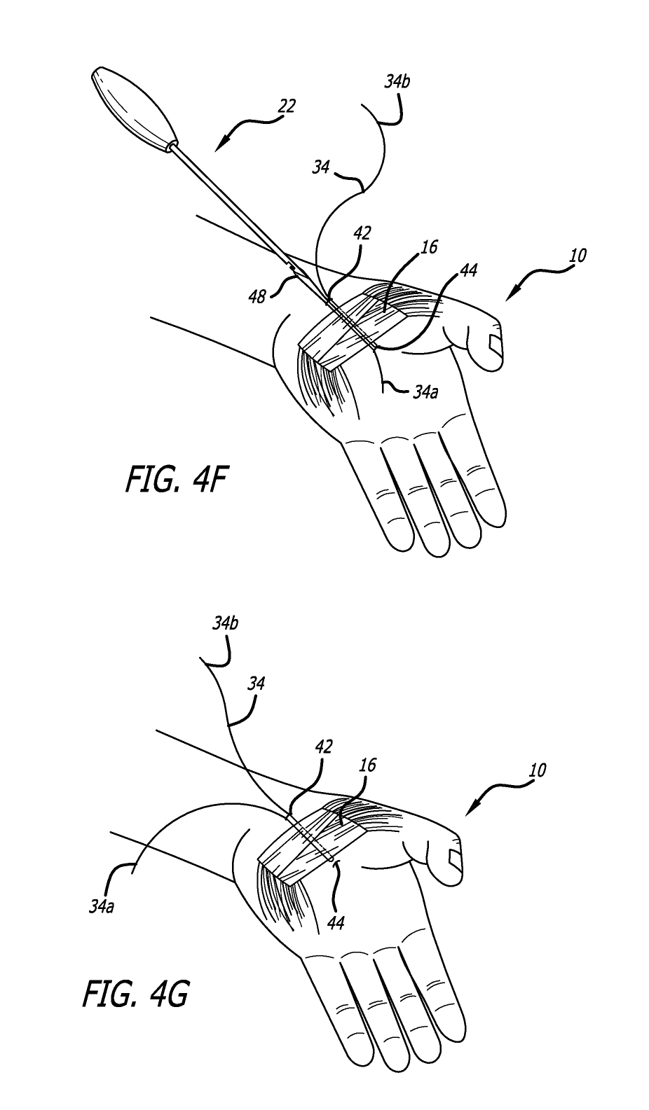

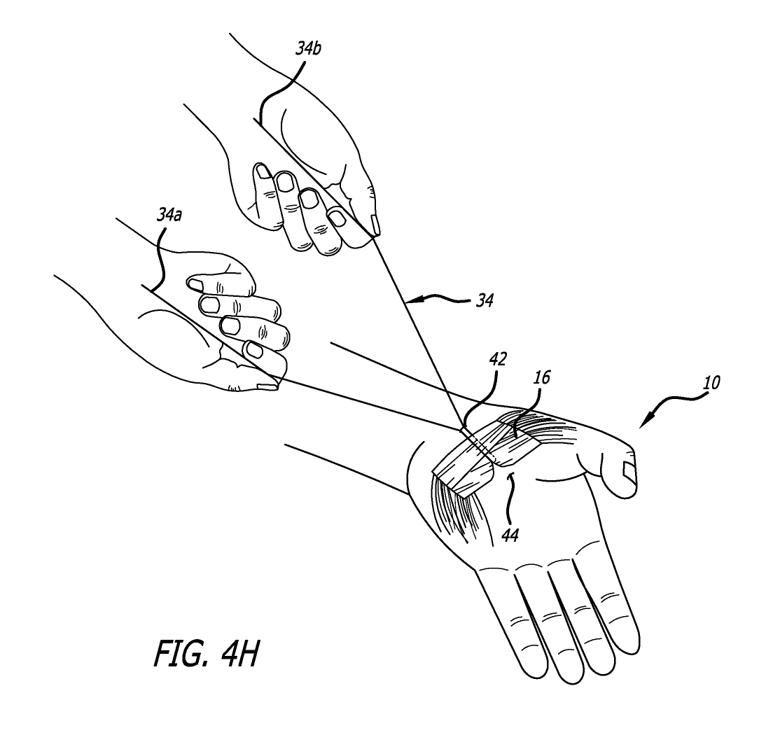

There are a number of ways to access the soft tissue to be transected. Such methods are fully described in my co-pending U.S. patent application Ser. Nos. 13/870,291 and 13/460,246. For purposes of illustrating the principles of my present invention, only a few particular methods will be disclosed herein. For example, FIGS. 4A-4H illustrate one preferred method of practicing the present invention. After anesthetizing the area of the hand 10 near and about the transverse carpal ligament 16, the distal end 30 of the retrieval needle 22 is brought into contact with the hand just proximal to the proximal edge of the target ligament as is shown in FIG. 4A. The ligament is visible in the Figures for purposes of clarity only as no incision is made throughout the entire procedure to in any way expose the ligament to view. Additionally, an imaging device, such as an ultrasound device, such as is commonly used for a variety of imaging applications, is used to visualize the position of the retrieval needle relative to the ligament but is not shown so as not to obscure the surgical site again for purposes of clarity. It is preferable to enter the hand at a position about 30 mm proximal of the proximal edge of the transverse carpal ligament as the carpal tunnel can then be entered at a shallower angle obviating the need to adjust the angle of the needle after the tunnel has been reached and thereby minimizing trauma to tissue in addition to allowing the retrieval needle to be more easily imaged.

In FIG. 4B, the retrieval needle has been advanced into the hand via entry port 42, through the carpal tunnel just under the ligament and out through exit port 44. The entry and exit ports may be formed by the direct extension of the retrieval needle through the skin in the event the retrieval needle 22 is selected to have a sharp distal tip 29. In the event a retrieval tool is used with a blunt tip, a sharp instrument is necessary for forming the access ports and guide the retrieval tool into the hand. The Figure additionally shows the cutting element 34 having been engaged in the hooking element 28 near the tool's distal end. In this particular embodiment, the cutting element is devoid of a locator tool attached to its distal.

Once the cutting element 34 is engaged, the retrieval needle 22 is retracted from the hand so as to draw a loop 46 of the cutting element into the hand via port 44, through the carpal tunnel and out of entry port 42 as is shown in FIG. 4C. The loop is then disengaged from the retrieval needle and while one end of the cutting element 34a is restrained, the loop is pulled so as to draw the opposite end 34b of the cutting element free of the hand as is shown in FIG. 4D.

FIG. 4E illustrates the subsequent step of the method wherein the retrieval needle 22 is readvanced into the hand via access port 42, is guided across the top surface of ligament 16 to remerge from the hand via access port 44. The section of cutting element 34 extending from under the ligament is engaged with the hooking element 28 of the retrieval needle.

Once the cutting element 34 is again engaged, the retrieval needle 22 is retracted from the hand so as to draw a loop 48 of the cutting element into the hand via port 44, through the carpal tunnel and out of entry port 42 as is shown in FIG. 4F. The loop is then disengaged from the retrieval needle and while end 34b of the cutting element is restrained, the loop is pulled so as to draw the end 34a of the cutting element free of the hand as is shown in FIG. 4G. The cutting element is thereby in position about ligament 16 for subsequent manipulation to effect the transection. As is shown in FIG. 4H, the ends 34a, 34b of the cutting element may simply be grasped by the user, may be wound around the hands or fingers of the user for a firmer grip or alternatively, may be fitted with handles to provide for maximum grip and control. Unequal forces can alternatingly be applied to the two ends of the cutting element to induce a reciprocating cutting action either by hand or with the use of an appropriately configured power tool. Alternatively, one end can be pulled with greater force than the other element so as to pull the cutting element in a single direction as it cuts through the ligament. As a further alternative, both ends can be pulled simultaneously with equal force to simply pull the cutting element through the ligament. When transection has been achieved, the cutting element is simply withdrawn through access port 42. Application of a small bandage over each of the access ports 42, 44 completes the procedure.

In an alternative embodiment, and as a modification to the step shown in FIG. 4C, the retrieval needle 22 is not completely withdrawn from access port 42. The needle is retracted just enough to expose the hooking element 28 and allow the loop 46 of the cutting element 22 to be disengaged and withdrawn, while most of the distal end 30 remains below the skin. As a result, it is more likely that the needle will follow the same pathway to the ligament 16 before traversing its top surface resulting in less trauma and disruption to intervening tissue both while advancing the needle as well as at the completion of the transection step.

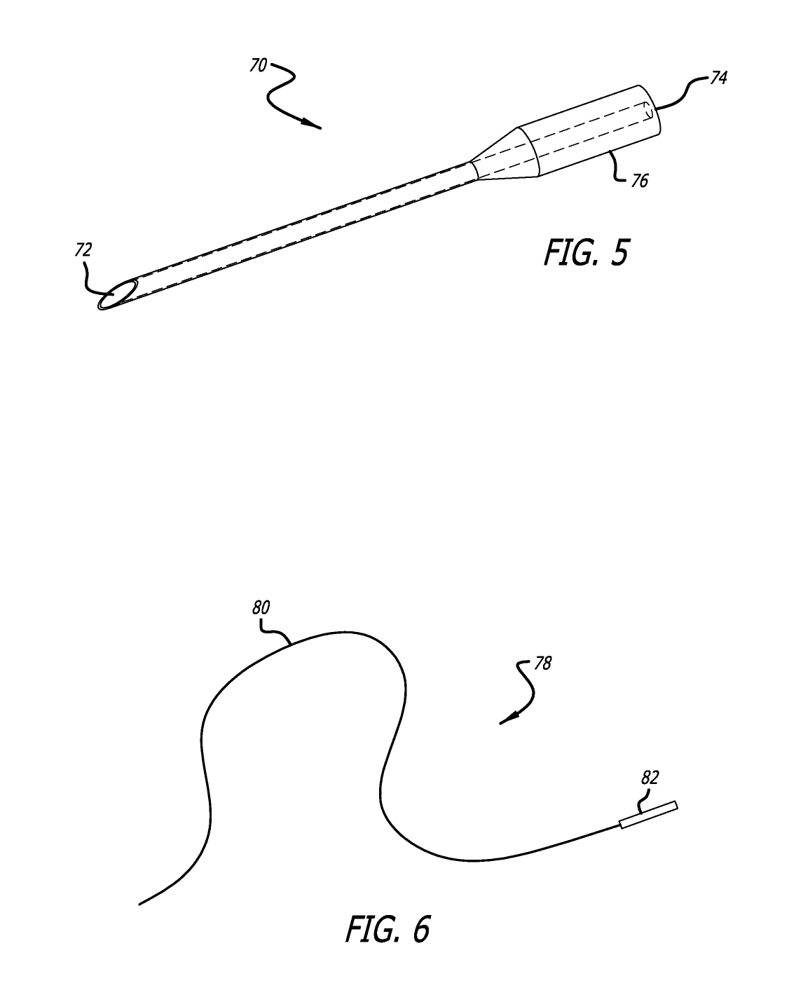

FIG. 5 is perspective view of another preferred embodiment of routing tool component of the present invention wherein the tool takes the form of a hollow introducer needle 70. The hollow needle includes a sharp or blunt distal end 72 and has hollow interior extending from its distal end to its proximal end 74. A handle 76 may be disposed about its proximal section to facilitate its manipulation. The length of the section of introducer needle distal to the handle is selected to be greater than the width of the target transverse carpal ligament. Its diameter is selected to be no greater than about 2 mm.

FIG. 6 is a perspective view of a preferred embodiment of the cutting element 78 of the present invention. Substantially the entire length 80 of the cutting element has a flexible, small diameter, thread-like structure with a high breaking strength and a smooth surface. The cutting element may comprise a monofilament or a plurality of braided, twisted or otherwise joined fibers or strands wherein each strand has a smooth surface so as to present a relatively smooth, none-abrasive surface. Its physical characteristics include a bend radius of less than half the thickness of the ligament and preferably a zero bend radius, a diameter of less than about 1.0 mm, and a breaking strength of over 2 lbs. The cutting element may comprise fiber or yarn formed of cotton, silk, glass fiber, carbon fiber, various plastic fibers or metal. More particularly, textile fiber, synthetic fiber, mineral fiber, polymer fiber, microfibers may be used. At least one end of the cutting element has a stiffened section 82 to facilitate the introduction into and the extension through the hollow introducer needle 70. The stiffened section may be formed by covering the section with relatively stiff tubing, by coating with a glue or a stiffening agent, by subjecting a synthetic fiber to heat, by the infusion of, for example, a resin or by the attachment of, for example, a suture needle. The stiffened section 82 preferably has a diameter less than the inner diameter of the introducer needle. The enhanced diameter shown in the drawing is for illustration purposes only.

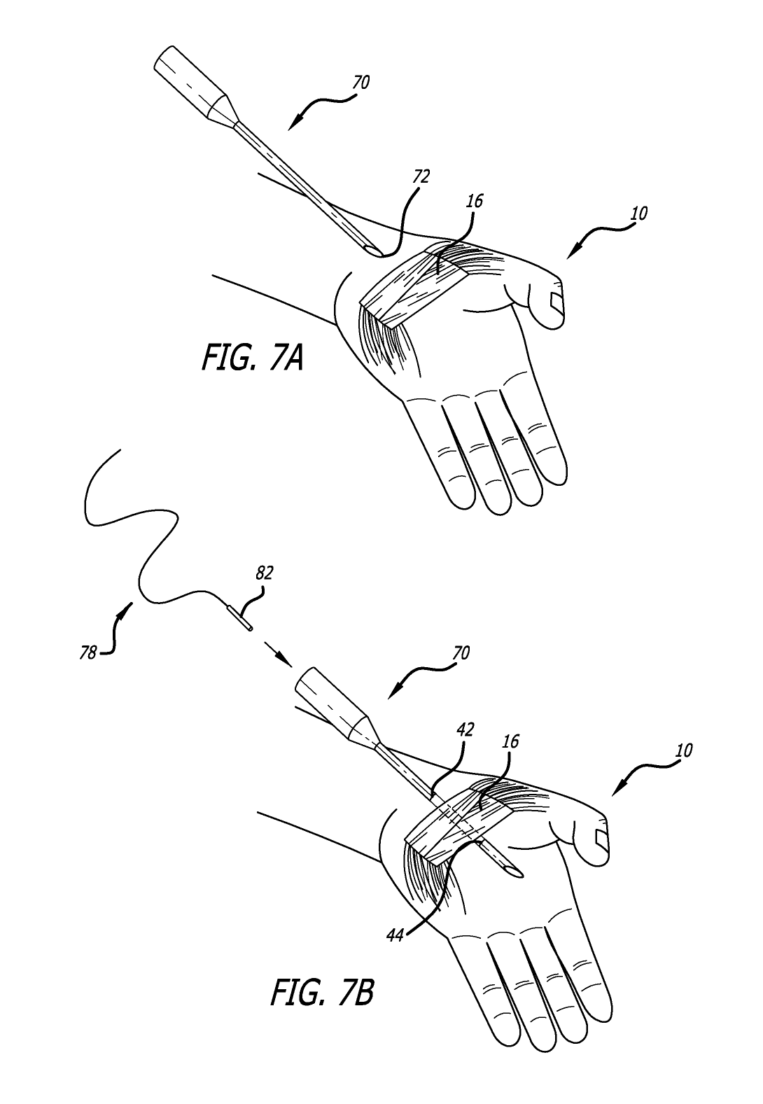

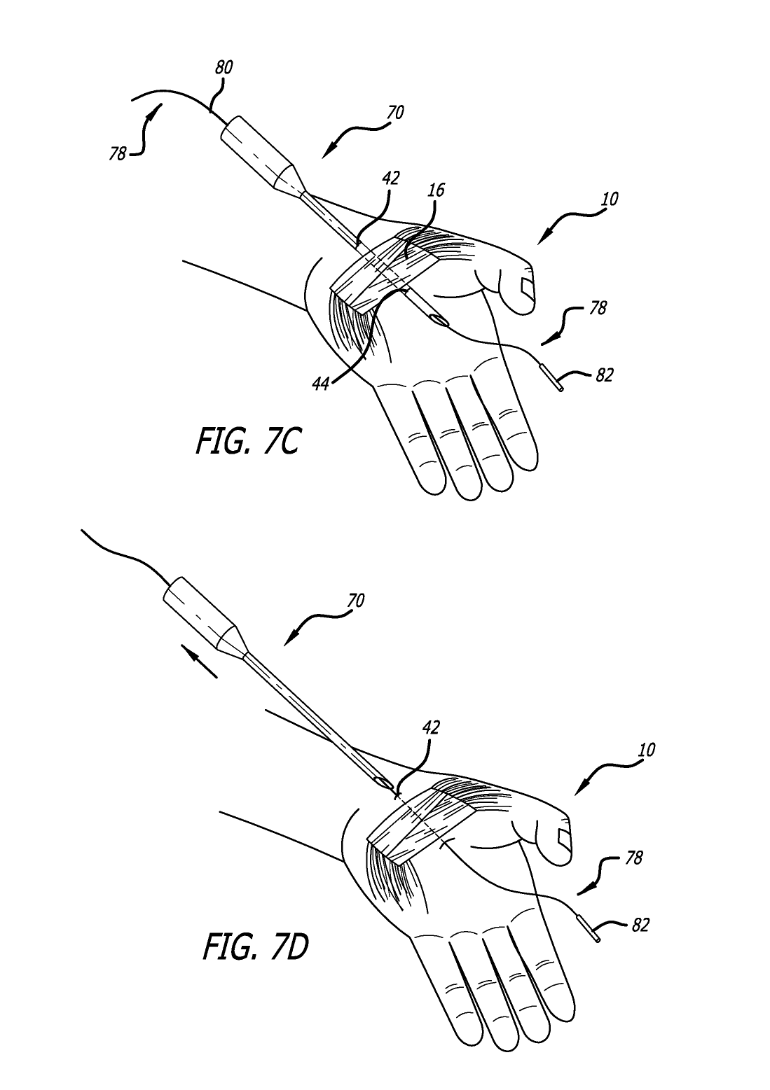

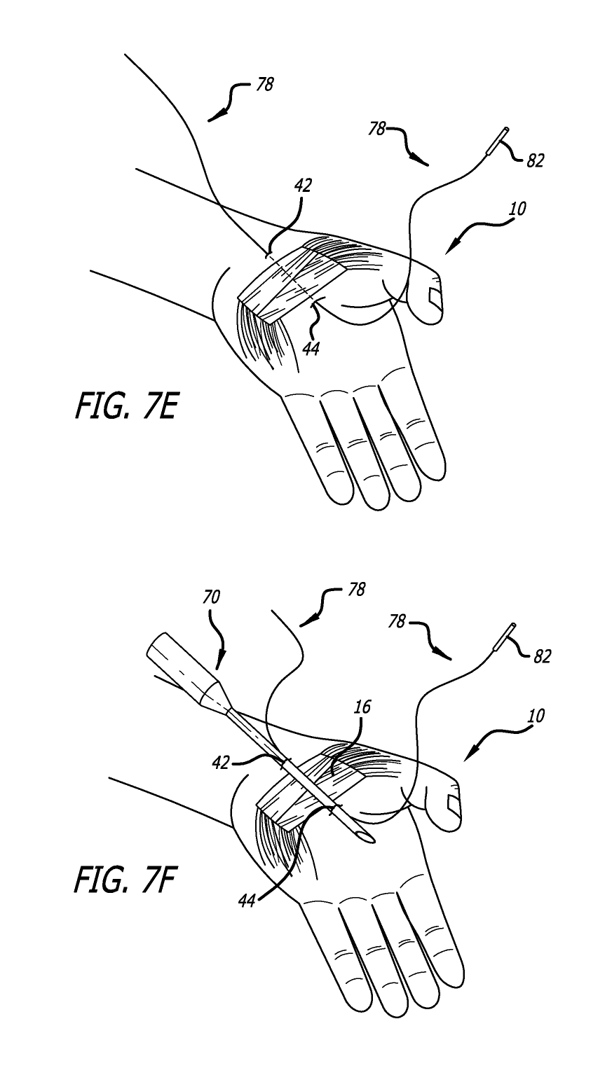

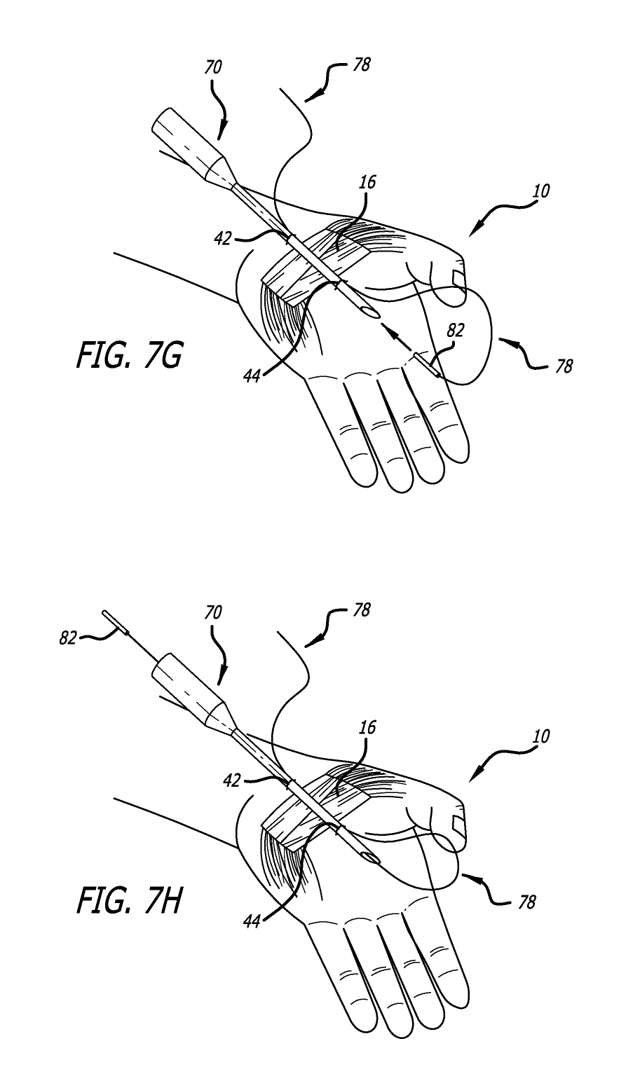

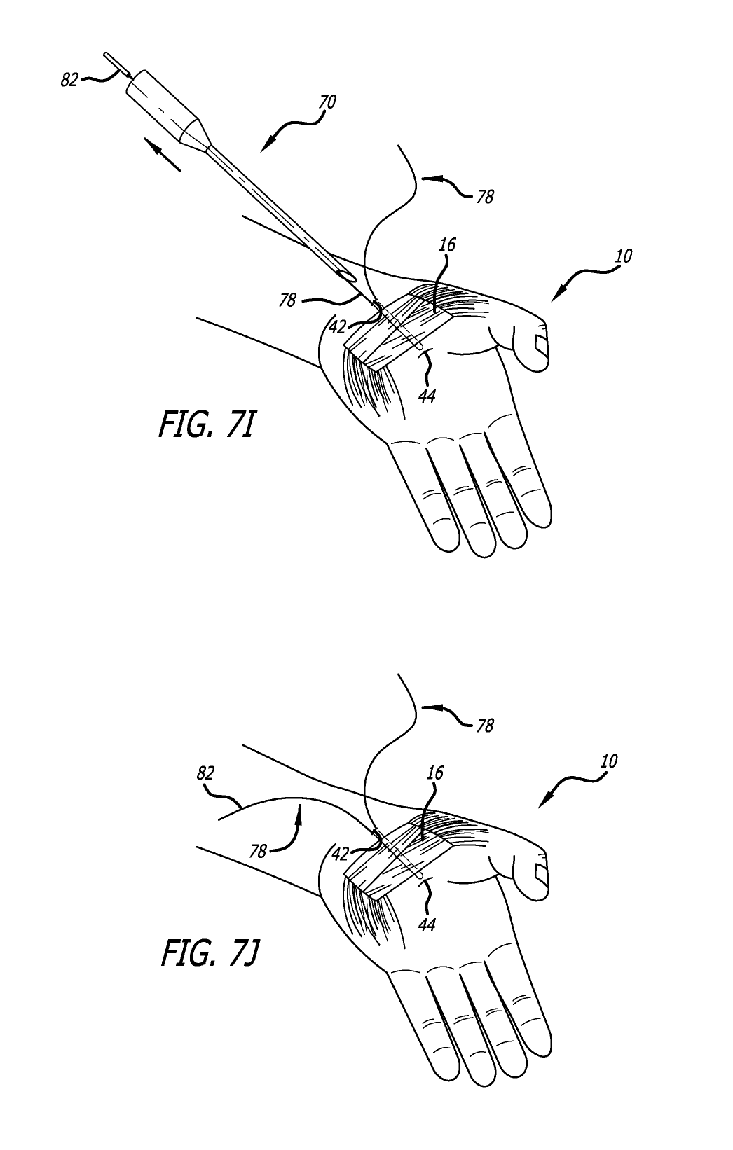

FIGS. 7A-J illustrate a preferred method of practicing the present invention. After anesthetizing the area of the hand 10 near and about the transverse carpal ligament 16, the distal end 72 of the hollow introducer needle 70 is brought into contact with the hand just proximal to the proximal edge of the target ligament as is shown in FIG. 7A. The ligament is visible in the Figures for purposes of clarity only as no incision is made throughout the entire procedure to in any way expose the ligament to view. Additionally, an imaging device, such as an ultrasound device, such as is commonly used for a variety of imaging applications, is used to visualize the position of the introducer needle relative to the ligament but is not shown so as not to obscure the surgical site again for purposes of clarity. It is preferable to enter the hand at a position about 30 mm proximal of the proximal edge of the transverse carpal ligament as the carpal tunnel can then be entered at a shallower angle obviating the need to adjust the angle of the needle after the tunnel has been reached and thereby minimizing trauma to tissue in addition to allowing the introducer needle to be more easily imaged.

In FIG. 7B, the introducer needle has been advanced into the hand via entry port 42, through the carpal tunnel just under the ligament and out through exit port 44. The entry and exit ports may be formed by the direct extension of the introducer needle through the skin. The Figure additionally shows the cutting element 78 being advanced toward the proximal opening of introducer needle wherein the stiffened section 82 of the cutting element serves to facilitate the threading of the cutting element into the needle's hollow interior.

FIG. 7C shows the cutting element emerging from the introducer needle's distal end while FIG. 7D illustrates the subsequent retraction of the needle to leave the cutting element in place as is shown in FIG. 7E. As such, a section of cutting element 78 is left projecting from entry port 42 and from access port 44 while its central section extends through the carpal tunnel just below the transverse carpal ligament 16.

FIG. 7F illustrates the subsequent step of the method wherein the introducer needle has been reintroduced into the hand via entry port 42 immediately adjacent to the placed cutting element 78. The introducer needle has been advanced through the hand immediately above the transverse carpal ligament 16 to reemerge from access port 44. Alternatively, the introducer needle may be reintroduced into the hand via access port 44 to reemerge from port 42.

Once the introducer needle 70 is again in place, the cutting element 78 is fed into the distal end of the introducer needle, is extended along the needle's hollow interior to project from its proximal end as is shown in FIG. 7H. Subsequent retraction of the introducer needle as per FIG. 7I leaves the cutting element in position about the ligament 16 as is shown in FIG. 7J. The cutting element is thereby in position for subsequent manipulation to effect the transection of the ligament.

Alternatively, a cutting element having a stiffened section at both ends allows the cutting element to be initially introduced into the distal end of the introducer needle and extended there through. After retraction of the needle and reintroduction into the hand and extension above the ligament to re-emerge from the hand, the second stiffened end of the cutting element can be inserted into the distal end of the needle and extended there through. Subsequent retraction of the needle again leaves the cutting element in position for the transection.

Alternatively, it is desirable to first introduce a flexible guiding wire with certain stiffness (such as monofilament of Nylon) into the hand and position it about the ligament in the manner as was described above with regard to placement of the actual cutting element. Once such guiding wire is in place, one end is attached to one end of the cutting element and simply pulled through so as to replace the guiding wire with the cutting element. Such approach does not require the flexible cutting element has a stiffed section at any end.

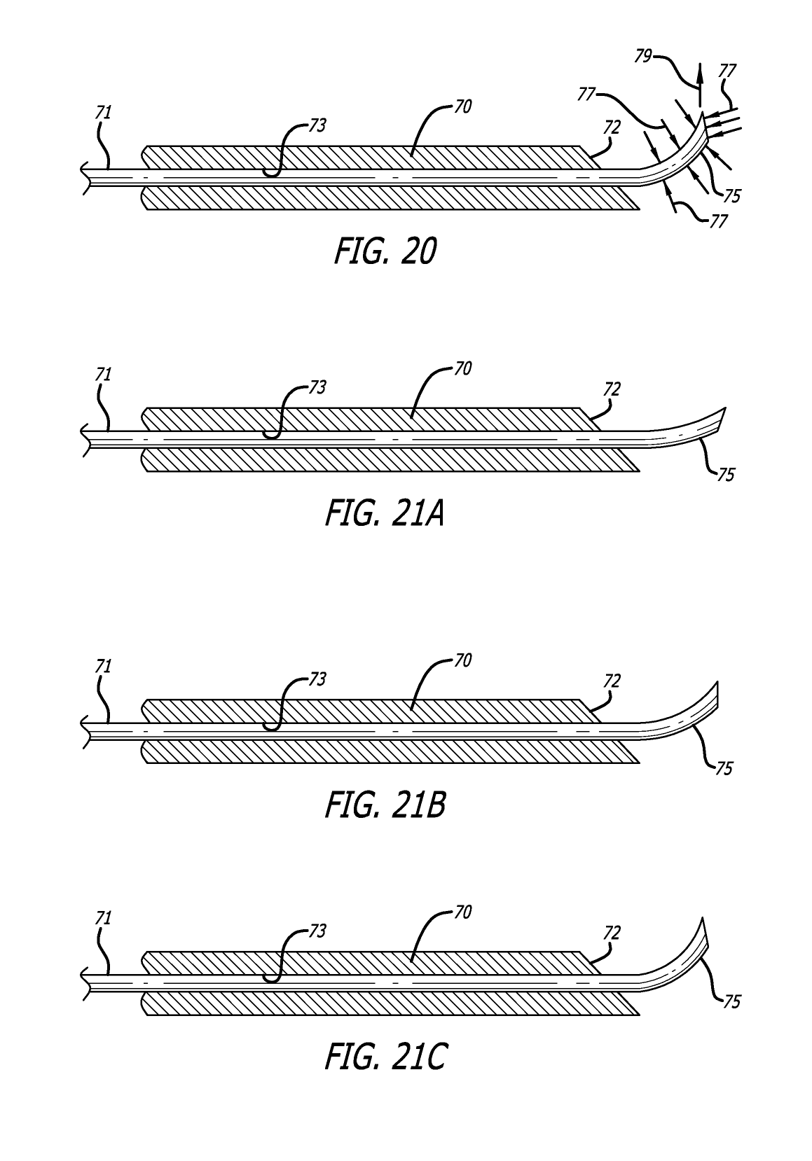

Alternatively, as is shown in FIG. 20, the introducer tool can comprise a first needle 70 with an internal lumen 72 and a flexible inner needle 71 capable of facilitating insertion into and extension through the internal lumen 73 of the first needle 70 to guide the direction of the flexible inner needle 71. The tip 75 of the flexible needle 71 is configured to be able to change direction under contact forces when the tip 75 extends out of the first needle 70 when placed in the body. FIG. 20 shows how contact forces (shown by arrows 77) can be applied to the tip 75 as it extends out of the lumen 73 of the hollow needle 70 into the desired direction (depicted by arrow 79).

The inner needle 71 also can be made of Nitinol alloy or other shape-memory alloys. FIGS. 21A-21C shows an inner needle 71 made from a Nitinol alloy and the gradual shapes it can take once placed within the patient's body. The needle 71 has a pre-memory-shaped curve at the tip 75 and is kept in a substantially straight shape (FIG. 21A) below a transition temperature assigned to be a body temperature. When the first needle 70 is placed inside the patient's body and needs to be a guiding, the inner needle 71 is inserted into the lumen 73 of the first needle 70 and extends out of the first needle 70 for designed length. The heat of the patient's body causes the tip 75 to gradually move (as is shown in FIG. 21B) to the original curved shape (depicted in FIG. 21C) so as to guide the tool in the desired direction.

In use, the cutting element 78 may simply be grasped by the user, may be wound around the hands or fingers of the user for a firmer grip or alternatively, may be fitted with handles to provide for maximum grip and control. Unequal forces can alternatingly be applied to the two ends of the cutting element to induce a reciprocating cutting action either by hand or with the use of an appropriately configured power tool. Alternatively, one end can be pulled with greater force than the other element so as to pull the cutting element in a single direction as it cuts through the ligament. As a further alternative, both ends can be pulled simultaneously with equal force to simply pull the cutting element through the ligament. When transection has been achieved, the cutting element is simply withdrawn through access port 42. Application of a small bandage over each of the access ports 42, 44 completes the procedure.

The cutting element may simply be grasped by the user, may be wound around the hands or fingers of the user for a firmer grip or alternatively, may be fitted with handles to provide for maximum grip and control. Alternatively, the ends of the cutting element can be attached to a power tool (described below) which produces a uniform pulling force that achieves a clean and true transection. Unequal forces can alternatingly be applied to the two ends of the cutting element to induce a reciprocating cutting action either by hand or with the use of a power tool. Alternatively, one end can be pulled with greater force than the other element so as to pull the cutting element in a single direction as it cuts through the ligament. As a further alternative, both ends can be pulled simultaneously with equal force to simply pull the cutting element through the ligament. When transection has been achieved, the cutting element is simply withdrawn through access port 42. Application of a small bandage over each of the access ports 42, 44 completes the procedure.

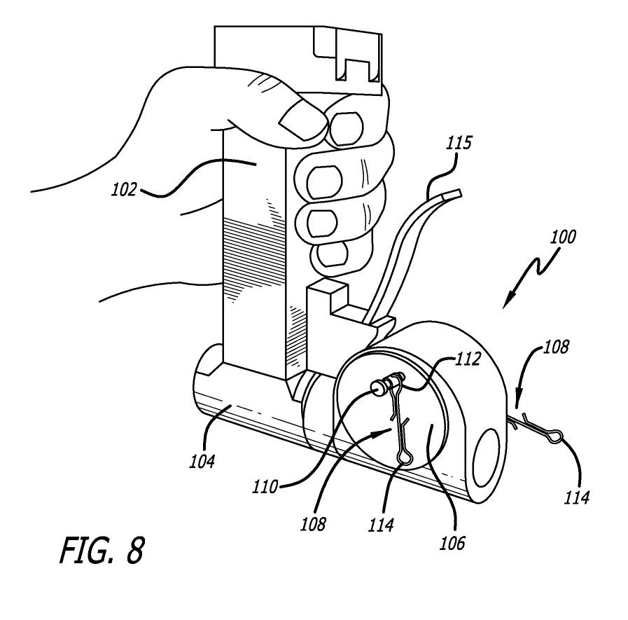

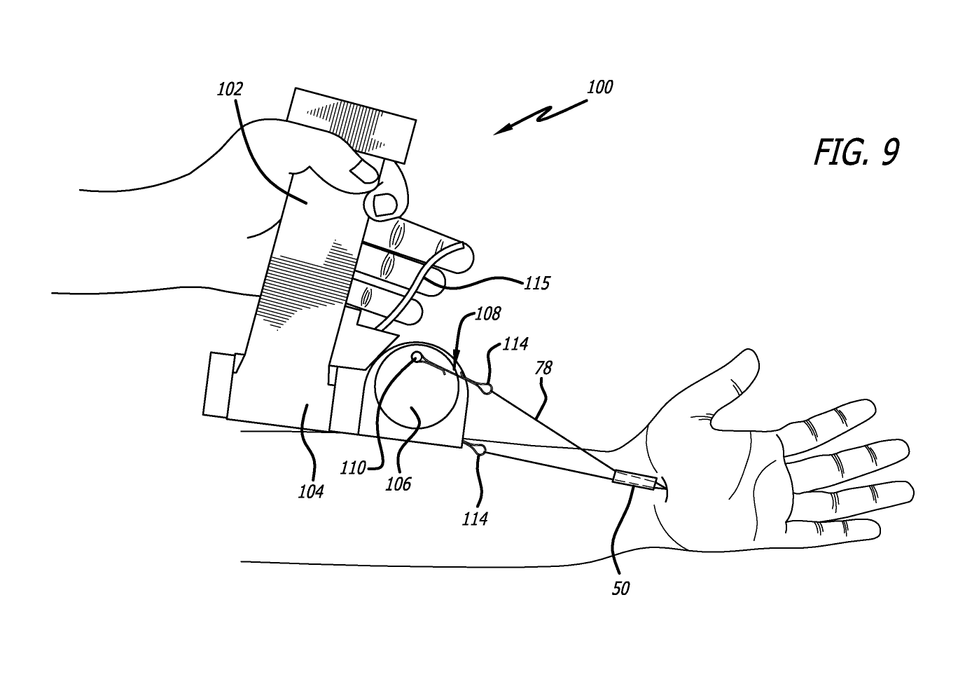

FIGS. 8 and 9 show an embodiment of a power tool 100 for providing the alternating releasing-pulling action on the cutting element 78. This power tool 100 includes a hand grip section 102, which may house a battery pack (not shown). An electric motor (not shown) would be placed within the housing or body 104 of the power tool 100, which is coupled to a conversion mechanism that develops the alternating releasing-pulling action. In the embodiment shown, the alternating releasing-pulling action is achieved by the rotation of a crankshaft (not shown in FIG. 8) which rotates a pair of rotatable discs 106 on each side of the device (only one of which is shown in FIG. 8). A pair of linking pins 108 are, in turn, rotatably connected to these rotating discs 106. A crankpin 110 is connected to each of the rotating discs 106 and is rotated as the disc 106 rotates. Each linking pin 108 has one end 112 which is rotatably connected to a crankpin 110 of each rotating disc 106 allowing the linking pin 108 to rotate about that end 112 while the disc 106 rotates. Each linking pin 108 includes a free end 114 to which the cutting element 78 can be attached (see FIG. 9). The alternating releasing-pulling action is achieved by allowing one of the linking pin 108 to be moved in one direction (via rotation its associated disc 106) while the other linking pin 108 moves simultaneously in substantially the opposite direction (again via rotation of its associated disc). The rotation of the linking pins 108 about the crankpins 110 associated with each rotating disc 106 achieves the alternating releasing-pulling movement which is then imparted to the cutting element 78 causing the portion of the cutting element in contact with the body tissue to start to transect the body tissue. A slight retraction of the power tool by the surgeon (while the ends of the cutting element are being reciprocated) causes a slight tension between the cutting element tissue to create an appropriate cutting action needed to transect the tissue.

The power tool 100 includes a power switch 115 which can be easily activated by the user. The power switch 115 can be selected to provide variable rotational speeds to the rotating discs 106 which in turn produce variable reciprocating speeds that are transmitted to the ends of the cutting element. For example, the speed of rotation of the rotating discs can be varied from about 10 to 5000 RPMs. The speed of rotation of the discs 106 and the developed reciprocating speed of the cutting element 78 can thus be easily varied by simply changing the position of the power switch 114 by the user.

The position of the linking pins 108 on each rotating disc 106 must be such that an effective reciprocating movement can be generated as the discs 106 rotate. In this regard, the crankpin 110 should be placed on its rotating disc 106 about 180° opposed to the position of other crankpin 110 on the other rotating disc 106. In other words, when one looks directly at the position of the crankpin 110 on one rotating disc 106, the other crankpin 110 on the other rotating disc 106 should be disposed about 180° from the other. This positioning of the crankpins 110 on the each disc 106 could maximize the reciprocating stroke that will be developed by the power tool. The diameter of each rotating discs 106 and the positioning of each crankpin 110 can be set to achieve the length of the reciprocating stroke. The power tool can develop a designed distant of a cutting stroke in the range of about 1 mm to 250 mm and a designed frequency in the range of about 10 to 10000 times per minutes.

FIG. 9 illustrates the power tool 100 being used to reciprocate the cutting element 78 that has been placed about the transverse carpal ligament. A protective sleeve 50 may be fitted so as to maintain the cutting element in alignment and to minimize trauma to the surrounding tissue. Although not shown in FIG. 9, this protective sleeve 50 could be held in place against the patient by using a sterile adhesive tape or bandage. As can be seen in FIG. 9, the power tool 100 allows the user to grip the device while maintaining the linking pins 108 close to the arm of the patient. This structure allows the cutting element 78 to remain closer and more parallel to the plane of the ligament that is being transected. Accordingly, the cutting action of the cutting element 78 on the ligament should be cleaner and truer than if the reciprocating linking pins 108 were placed above the plane of the ligament.

In a particular method of usage, the cutting element 78 is initially placed over the tissue of interest using any of the methods described above. In the procedure depicted in FIG. 9, the cutting element 78 would be initially placed around the transverse carpal ligament of the patient utilizing the techniques described above. Thereafter, each end of the cutting element 78 would be attached to one of the free ends 114 of the linking pins 108. The free end 114 of the linking pin 108 has a structure which allows the user to quickly wrap the cutting element 78 about the end 114. The surgeon would then move the power tool 100 close to the patient's arm and take up any slack in the cutting element 78. The surgeon would then apply force to the power switch 115 to start the reciprocating action needed to cut the ligament. The surgeon would then carefully place the needed amount of force on the power switch to obtain the desired speed of the reciprocating action. As the reciprocating action causes the cutting element 78 to move back and forth, the surgeon would then gently retract the power tool 100 creating some tension between the cutting element 78 and ligament. The surgeon would then continue to carefully retract the power tool allowing the cutting action of the cutting element 78 to completely transect the ligament. Once the ligament has been completely transected, the cutting element 78 will no longer be engaged with the ligament and should be able to be carefully removed from the opening in the patient. The small openings placed in the patient in order to properly position the cutting element 78 around the ligament can then be covered by a bandage.

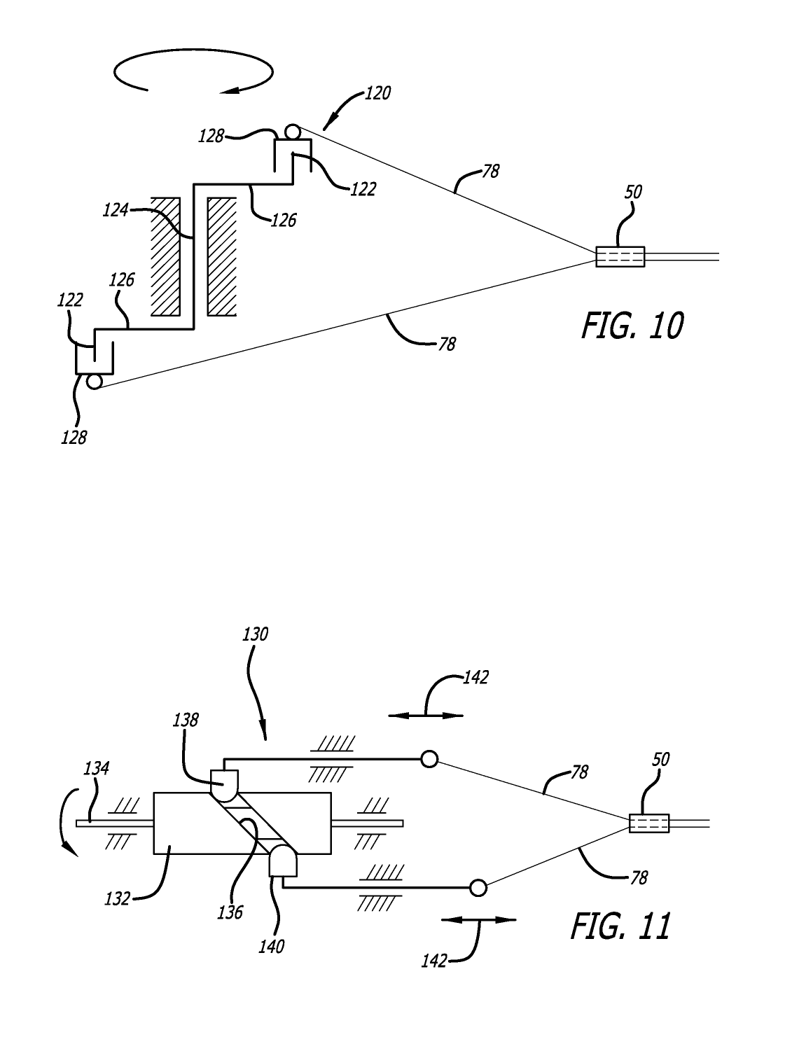

The power tool 100 utilizes just one of a number of mechanisms that can be used to develop the necessary alternating releasing-pulling action on the cutting element 78. Generally, the power tool 100 would utilize an electric drive motor that produces rotation to a shaft. Converting the rotation of a longitudinally positioned electric motor to a transversely disposed shaft can be achieved in any of various mechanisms including for example geared, cammed or desmodromic mechanisms, among many others. FIGS. 10-14 show a number of different mechanisms that can be used with the motor drive of the power tool to develop the alternating releasing-pulling action. It should be appreciated that although not shown, each of the actuating mechanisms shown in FIGS. 10-14 can be rotated, for example, by the motor drive associated with the power tool. Each actuating mechanism could be housed within the body of the power tool 100.

Referring initially to FIG. 10, there is shown a schematic representation of an embodiment of a mechanism 120 made in accordance with the present invention which utilizes two crankpins 122 which can be rotated to develop the alternating releasing-pulling action. A rotatable shaft 124 with oppositely extending members 126 are connected to the crankpins 122 which in turn have rotatable components 128 that are attached to the cutting element 78. This particular mechanism 120 operates much like the pedals of a bicycle in that the rotatable members 128 rotate as the shaft 124 rotates. Although not shown, a linking pin 108 could be attached to each rotatable component 128. The action developed by this mechanism 120 produces a uniform movement of the cutting element 76 which is shown extending through a protective sleeve 50 and would be place around the target tissue (not shown).

Referring now to FIG. 11, an alternative actuating mechanism 130 for developing the alternating releasing-pulling action is shown. This mechanism 130 a large rotatable cylinder 132 which is connected to and is rotated by the crankshaft 134 of the drive motor of the power tool. As can be seen in FIG. 11, the rotating cylinder 132 includes a groove or channel 136 cut into the outer surface of the cylinder 132. This channel 136 is designed to receive and move a pair of cam followers 138 and 140 which move in a substantial linear movement as the cylinder 132 rotates. This substantial linear motion, in turn, is transmitted to the cutting element 78. This channel 136 extends in a helical pattern around the cylinder 132 and reconnects to itself to create a closed loop. Arrows 142 show the linear movement of the pair of followers 138 and 140. It should be appreciated that the cam followers 138 and 140 would have to be kept restrained within a device (schematically shown) that restricts each cam follower 138 and 140 to follow a linear movement as the cylinder 132 rotates. The cam followers 138 and 140 can, in turn, be attached to linking pins 108 (not shown) similar to the ones shown in FIGS. 8 and 9. The speed of the alternating releasing-pulling action of the cam followers can be varied by simply changing the rotational speed of the cylinder 132. The reciprocating stroke can be varied, for example, by changing the longitudinal length of the channel 136.

FIG. 12 shows another actuating mechanism 150 that could be used to develop the alternating releasing-pulling action on the cutting element 78. As can be seen in FIG. 12, a pair of cams 152 and 154 connected to a pair or followers 156 and 158 can be used to develop the linear releasing-pulling action (depicted by arrows 142) that acts on the cutting element 78. A connecting member 160 can be used to connect each of the cams 152 and 154 together. The rotation of the cams 152 and 154 are depicted by arrows 162. Accordingly, utilizing this particular mechanism, only a single cam needs to be rotated since the other cam will rotate accordingly via the connection via the connecting member 160.

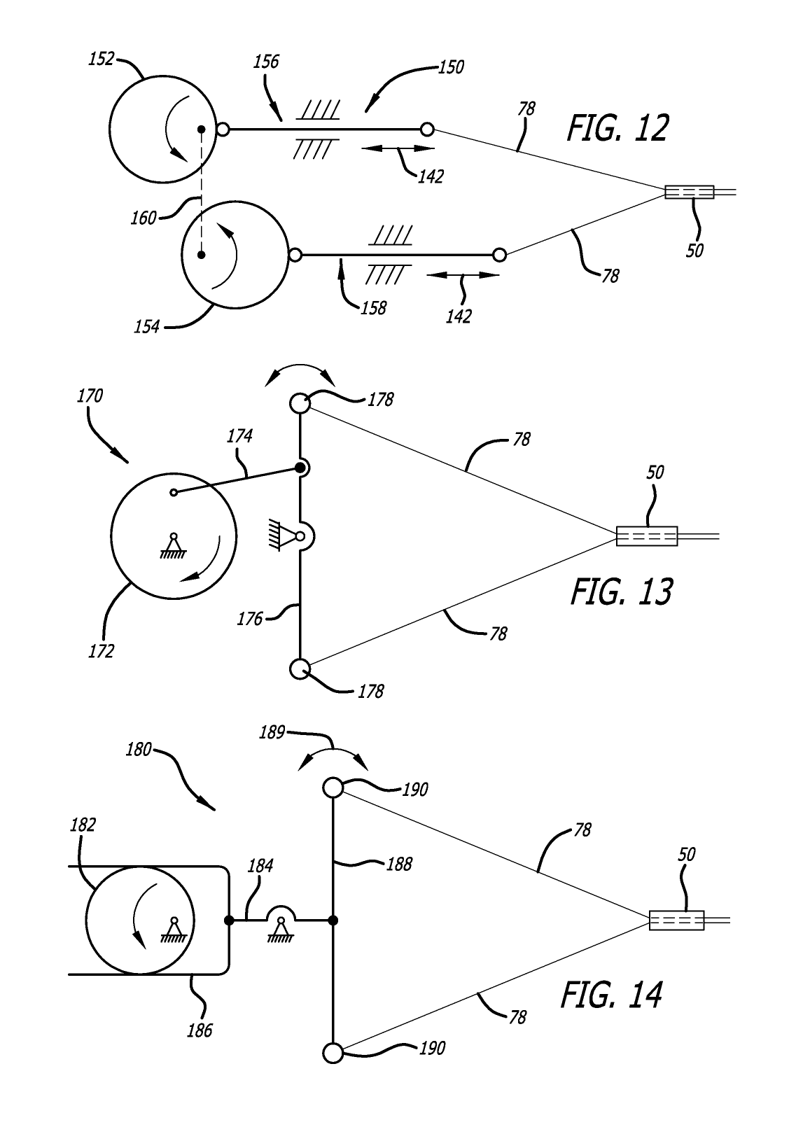

Another actuating mechanism 170 is depicted schematically in FIG. 13. This particular mechanism utilizes a 4-bar linkage to produce a rocking arm arrangement to develop the alternating releasing-pulling action on the cutting element 78. As can be seen in FIG. 13, only a single crankpin is needed to move the cutting element in the alternating releasing-pulling action. The cam 172 is rotated and is connected with a bar linkage 174 which is, in turn, connected to a rocking arm assembly 176. As the cam 172 rotates, the bar linkage 174 moves the rocker arm assembly 176 causing both ends 178 of the assembly 176 to move in opposite directions from each other. As a result, the attached cutting element 78 will be subject to an alternating releasing-pulling action which will develop the cutting action on the target tissue.

FIG. 14 shows a further actuating mechanism 180 which also utilizes a single cam 182 to move the cutting element 78. as can be seen in FIG. 14, the mechanism 180 includes a rocking arm 184 which includes a fork-like member 186 which contacts the cam 182. As the cam 182 rotates in the direction depicted by arrow 162, the fork-like member 186 causes the rocking arm 184 to move and pivot causing arm 188 to move in a direction depicted by arrow 189. The ends 190 of the arm 188, which are attached to the cutting element 78, move to produce the alternating releasing-pulling motion on the cutting element 78. Alternatively, the function of the power tool can also be achieved by a manually operated hand tool powered by the mechanism of a hand-turning crank and designed with the actuating mechanisms described above.

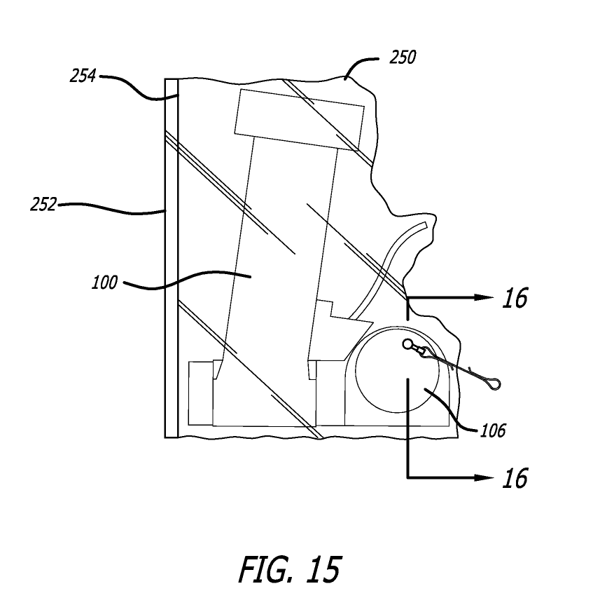

Referring now to FIGS. 15-19, a disposable, sterilized isolative system is shown encasing an embodiment of the power tool 100. This isolative system is shown embodied as a protective bag 250 which allows the power tool 100 to be used numerous times before the power tool 100 has to be re-sterilized. The protective bag 250 is disposable after each use so that a new protective bag 250 could be easily placed on the power tool 100 after each patient use. The bag 250 can be made from a soft plastic material or plastic-like material which is impermeable to germs and/or bacteria. The protective bag 250 would be made with a thin wall so that the bag 250 is soft and flexible. The protective bag 250 thus forms a protective barrier to germs and bacteria while allowing the user to properly hold the power tool and actuate the power switch.

The bag 250 has a main body including large opening 252 which can be closed by a locking component associated with the bag. For instance, a re-sealable lock 254 can be a simple plastic “zipper” similar to those used, for example, in ZIPLOC® brand products. The re-sealable lock 254 basically utilizes a pair of mating components located on opposites sides of the bag and which are capable of being pressed into engagement with each other to create a suitable seal. The re-sealable lock could be “sliderless” which allows the user to simply apply a pressure on the mating components to lock the components in place. Alternatively, the re-sealable lock 253 could utilize a “slider” which can held by the user and run along the mating components to apply the pressure needed to lock the mating components together. Alternative locking components would include adhesive tape, rubber band and other suitable devices which could be used as well without departing from the spirit and scope of the present invention. Alternatively, the opening 252 could be heat sealed closed after the power tool is placed within the protective bag 250.

The locking component 254 is shown in FIG. 15 near the opening 252 of the protective bag 250 in order to properly seal the bag 250 during usage. Generally, the locking component 254 extends the same length as the opening 252. This large opening 252 allows the power tool 100 to be easily inserted and removed from the bag 250. Also, as can be seen in FIG. 15, the protective bag 250 can be formed to substantially match the outer profile of the power tool 100. Thus, the hand grip 102 and power switch 114 can be easily grasped and actuated by the user even with the protective bag 250 in place.

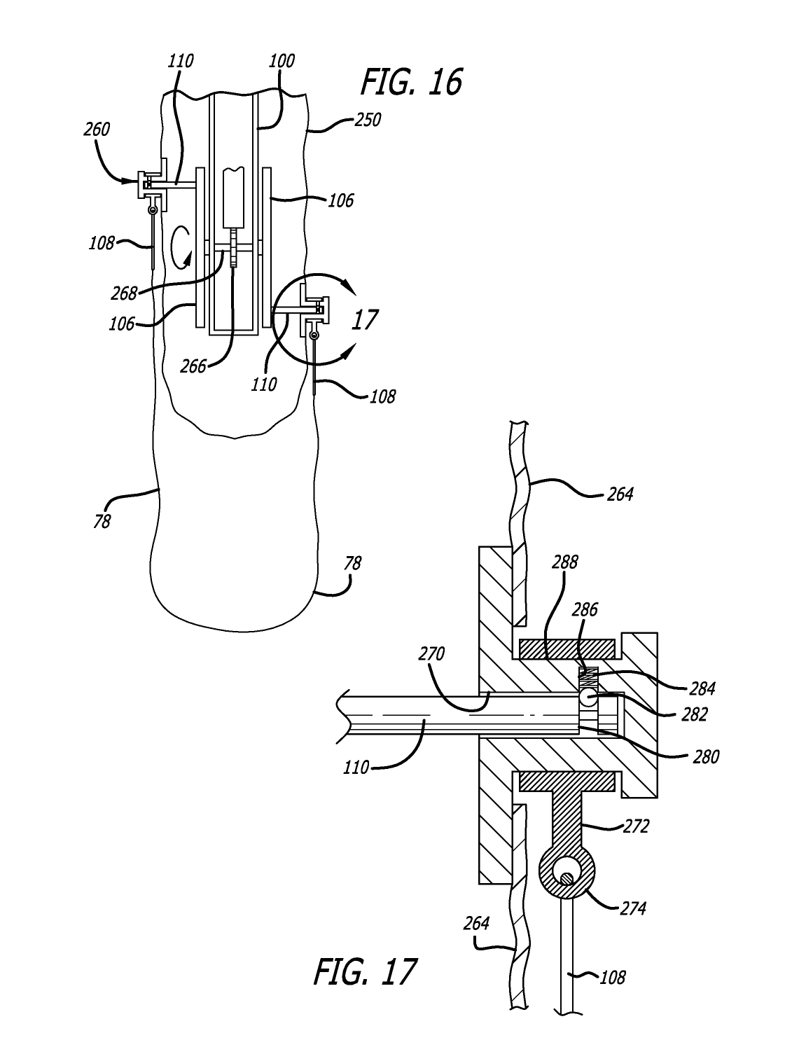

Referring now to FIG. 16, a cross sectional view of the moving components of the power tool is shown with respect to their placement in the protective bag 250. The particular power tool 100 shown in FIG. 16 utilizes a pair of rotating discs 106 and crankpins 110 as shown in FIGS. 8 and 9. Linking pins 108 (located outside of the protective bag 250) are attached to the cutting element 78. As can be seen in FIG. 16, the power tool 100 and the rotating discs 106 and crankpins 110 remain within the confines of the protective bag 250 during usage.

As can be seen in FIGS. 16 and 17, the protective bag includes a mechanism 260 which allows the alternating releasing-pulling action of the power tool to be transferred from the confines of the protective bag 250 to the outside of the bag 250 where it will be delivered to the cutting element 78. The mechanism 260 includes a pair of housings 262 configured to be separately held on outer portions of the power tool (via the crankpins 110). These housings 262 are formed with, or are attached to, the wall 264 of the protective bag 250 to maintain a hermetical seal. The power tool 100 is shown schematically as a gear 266 which is driven and connected to a shaft 268 that is rotated by a drive motor (not shown). The shaft 268 rotates as is indicated by the arrow in FIG. 16. Each end of the shaft 268 is attached to a disc 106 which, in turn, moves the crankpins 110. Each crankpin 110 is placed within an opening 270 formed on the housing 262 and is rotatable within the opening 270 of the housing. A holder 272 which includes a component such as an eyelet 274 is rotatable about the housing 262, as can be seen in FIG. 17. The eyelet 274 is, in turn, connected to the linking pin 108. As the crankpins 110 move with their rotating discs 106, the housing 262 moves as well allowing the holders 272 to move with the crankpins 110. Accordingly, the linking pins 108 also move allowing the alternative releasing-pulling action to be placed on the cutting element 78.

FIG. 17 shows a particular arrangement of components which help to prevent the housing 262 from bunching or tearing as the crankpins 110 move. The end of the crankpin 110 may include an annular channel 280 which is designed to accept an ball bearing 282 associated with the housing 262. The ball bearing 282 is biased outwardly by a biasing element such as a spring 284 which is housed within a recess 286 formed in the housing 262. When the housing 262 is moved by the crankpin 110, the ball bearing 282 rolls within the channel 280 maintaining coupling between the crankpin and housing 262 while preventing the housing from spinning from the movement of the crankpin 110. These components produce a simple mechanism for maintaining the housing 262 mounted for movement with each crankpin 110. The housing also has a sliding outer surface 288 upon which the holder 272 can rotate about. The housing 262 may further include a cap 290 which prevents the holder 272 from moving off of the housing 262. This housing 262 with its associated components thus creates and provides a simple mechanism that will help prevent the protective bag from ripping should the rotating disc 106 engage the surface of the bag while allowing the alternating releasing-pulling action to be applied to the cutting element 78.

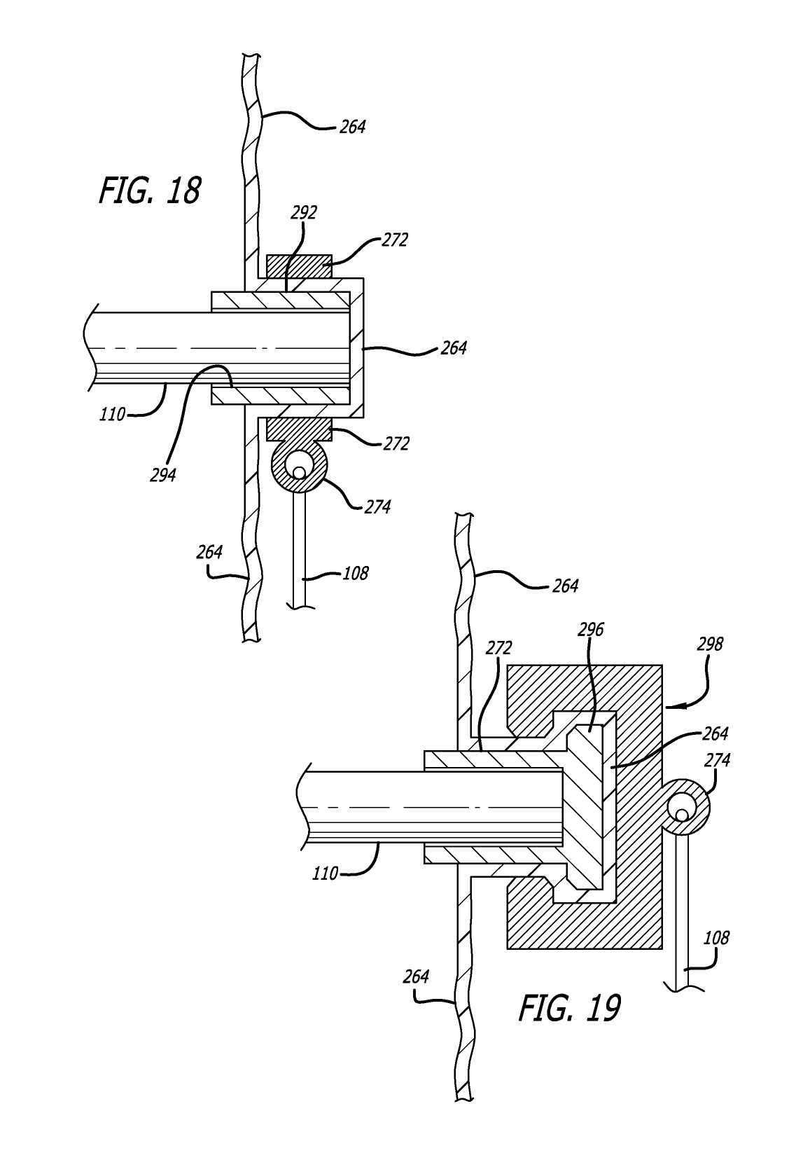

Referring now to FIG. 18, the crankpin 110 is shown inserted into a sleeve 292 which is found on the inside of the protective bag 250. This sleeve 292 basically replaces the housing 262 by allowing the crankpin 110 to extend within the lumen 294 of the sleeve 292 which is made from a material which allows the crankpin to freely slide therein. A holder 272 is then placed over the portion of the wall 264 of the protective bag 250 which extends over the sleeve 292. The holder 272 includes the eyelet 274 which is used to attach the linking pin 108 thereto. This arrangement allows the sleeve 292, holder 272 and linking pin 108 to move with the crankpin without binding or tearing. This structure ensures that a hermetical seal is maintained at the portion of the protective bag 250 where the crankpins 110 are moving.

Referring now to FIG. 19, an alternative structure to the one shown is FIG. 18 is shown. In this particular arrangement, the end of the crankpin 110 is shown inserted into a sleeve 292 which includes a cap 296. This sleeve 292 and cap 296 remain inside of the protective bag 250. A different holder 298 is utilized with the sleeve 272 and cap 296. As can be seen in FIG. 19, the holder 298 include an eyelet 274 which is used to hold the end of the linking pin 108. The holder 298 includes a structure which is adapted to receive the sleeve 272 and cap 296 with the portion of the bag wall 264 disposed therebetween. As the crankpin 110 rotates, the sleeve 272 acts as a contact surface which prevents any rotation of the crankpin 110 to be transferred to the holder 298. Rather, the holder 298 will move with the crankpin 110 but will not spin about the crankpin 110 as it spins. Accordingly, the alternating releasing-pulling action achieved by the crankpin and power tool will be transferred outside of the bag where it will be directed to the cutting element 78. This structure also ensures that a hermetical seal is maintained at the portion of the protective bag 250 where the crankpins 110 are moving.

The protective bag 250 may be made from a commercial grade plastic which is flexible to allow the bag to move without incurring a puncture by moving parts. The movable housing 262 can be made from a harder plastic which has greater rigidity than the flexible bag 250 to allow the locking components to properly engage without breakage. The movable housing 262 can be attached to the softer plastic of the main body of the bag 250 utilizing techniques known in the art such as heat bonding and adhesive techniques. Alternatively the isolative system described above is also applicable to a manual tool if the size of the bag is big enough to allow operating the hand-turning crank outside the bag.

While particular forms of the invention have been described and illustrated, it will also be apparent to those skilled in the art that various modifications can be made without departing from the spirit and scope of the invention. For example, the sequence of steps may be altered so as to cause the retrieval tool to traverse and then retrieve a loop of the cutting element across the top surface of the transverse carpal ligament before traversal of the bottom surface is achieved. Additional access ports may be formed for easier looping of the cutting element. Any of various ports can be used as the final exiting port of the two ends of the cutting element. Additionally, the method and appropriately dimensioned retrieval tool can be used to transect other tissue so as to perform for example, but not limited to, trigger finger release surgery, carpal tunnel release surgery, Achilles tendon extension surgery and plantar fascia release surgery. The apparatus and method can readily be adapted to transect other soft tissue such as for example muscle, tendon, vessels and nerves in humans as well as animals. Accordingly, it is not intended that the invention be limited except by the appended claims.

What is claimed:

1. A routing system for routing a flexible cutting element about a targeted soft tissue in a body in preparation of transection of the soft tissue using the flexible cutting element, comprising:
a routing tool configured to facilitate its extension into the body and routing the flexible cutting element about the soft tissue such that both ends of the flexible cutting element extend from a single access port in the body; and
an imaging device capable of visualizing the interior of the body.

2. The routing system of claim 1, wherein the imaging device comprises an ultrasound imaging device.

3. The routing system of claim 1, wherein said target soft tissue is a ligament or tendon.

4. The routing system of claim 1, wherein said target soft tissue is the transverse carpal ligament or annular ligaments of fingers or laciniate ligament of foot or transverse intermetatarsal ligament of foot or aponeurosis of foot or plantar fascia of foot or Achilles tendon of foot.

5. The routing system of claim 1, wherein the flexible cutting element has a zero bend radius.

6. The routing system of claim 1, wherein the routing tool comprises a unitary component.

7. The routing system of claim 6, wherein the routing tool is a needle-like member having a length sufficient to extend from a first location adjacent to the soft tissue to be transected to a second location adjacent to the soft tissue and opposite to the first location.

8. The routing system of claim 7, wherein the routing tool has a configuration for engaging the flexible cutting element and maintaining engagement therewith under a tensile load.

9. The routing system of claim 8, wherein the configuration of the routing tool includes a hook-shaped void.

10. The routing system of claim 8, wherein the configuration of the routing tool includes an eye-shape void.

11. The routing system of claim 7, wherein the routing tool is connected to the flexible cutting element.

12. The routing system of claim 7, wherein the routing tool has an internal lumen for receiving the flexible cutting element and the flexible cutting element has at least one stiffened end section to facilitate insertion into and extension through the internal lumen of the routing tool.

13. The routing system of claim 7, further comprising a flexible guiding wire capable of engaging the flexible cutting element and maintaining therewith under a tensile load, and wherein the routing tool has an internal lumen for receiving the wire.

14. The routing system of claim 1, wherein the routing tool is a hypodermic needle capable of injecting liquid into the body to hydraulically clear up the routing access about the soft tissue.

15. The routing system of claim 1, wherein the routing tool has a first needle with an internal lumen and a solid needle capable of facilitating insertion into and extension through the internal lumen of the first needle to guide the direction of the tool.

16. The routing system of claim 15, wherein said solid needle is elastic or flexible and has a tip configured to direct at a designed angle under the contact forces during routing.

17. The routing system of claim 15, wherein said solid needle is made of Nitinol Alloy or other shape-memory alloys, and pre-memory-shaped with a curved tip portion, and kept in a straight shape below a transition temperature assigned to be a body temperature; and capable of utilizing insertion into the first needle, and further extension out of the first needle for designed length, and returning the original curve shape at the tip portion at the body temperature for guiding the tool in designed direction.

18. A method for routing a flexible cutting element about a targeted soft tissue in a body in preparation of transection of the soft tissue using the flexible cutting element, comprising the steps of:
providing a routing tool;
extending the routing tool into the body and routing the flexible cutting element about the soft tissue such that both ends of the flexible cutting element extend from a single access port in the body; and
providing an imaging device and imaging tool relative to said soft tissue as said tool is used to rout said cutting element about said tissue.

19. The routing method of claim 18, wherein the imaging device comprises an ultrasound imaging device.

20. The routing method of claim 18, wherein said targeted soft tissue is a ligament or tendon.

21. The routing method of claim 18, wherein said target soft tissue is the transverse carpal ligament or the annular ligament of fingers or the laciniate ligament of foot or the transverse intermetatarsal ligament of foot or the aponeurosis of foot or the plantar fascia of foot or Achilles tendon of foot.

22. The routing method of claim 18, wherein the flexible cutting element has a zero bend radius.

23. The routing method of claim 18, wherein the routing tool comprises a unitary component.

24. The routing method of claim 23, wherein the routing tool is a needle-like member having a length sufficient to extend from a first location adjacent to the soft tissue to be transected to a second location adjacent of the tissue and opposite the first location.

25. The routing method of claim 24, wherein the routing tool has a configuration for engaging the flexible cutting element and maintaining engagement therewith under a tensile load, and wherein the steps further comprise:
extending the routing tool into the body at a first location adjacent to the soft tissue and out of the body at a second location adjacent of the tissue and opposite the first location so as to traverse the tissue on a first side thereof;
engaging the cutting element with the routing tool and re-tracking the routing tool to pull a first portion of the cutting element into the body at the second location and out of the body at the first location;
disengaging the first portion of the cutting element from the routing tool;
extending the routing tool from the first location through the body toward the second location so as to traverse the tissue on a second side thereof opposite the first side;
engaging the cutting element with the routing tool and re-tracking the tool to pull a second portion of the cutting element into the body at the second location and out of the body at the first location; and
disengaging the second portion of the cutting element from the routing tool so as to hereby leave the cutting element in place looped about the soft tissue with both end portions extending from the same location of the body.

26. The routing method of claim 25, wherein the configuration of the routing tool includes a hook-shaped void.

27. The routing method of claim 25, wherein the configuration of the routing tool includes an eye-shape void.

28. The routing method of claim 24, wherein the routing tool is connected to the flexible cutting element.

29. The routing method of claim 24, wherein the routing tool has an internal lumen for receiving the flexible cutting element and the flexible cutting element has at least one stiffened end section to facilitate insertion into and extension through the internal lumen of the routing tool, and wherein the steps further comprise:
extending the routing tool into the body at a first location adjacent to the soft tissue and out of the body at a second location adjacent of the tissue and opposite the first location so as to traverse the tissue on a first side;
extending the cutting element through the routing tool to leave a middle portion of the cutting element received by the routing tool;
re-tracking the tool from the body so as to leave the cutting element in place;
extending the routing tool into the body at the first location and out of the body at a second location so as to traverse the tissue on a second side and opposite the first side;
extending the cutting element from the distal end to the proximal end of the cutting element while keeping the in-body portion of the cutting element in place; and
re-tracking the tool from the body so as to hereby leave the cutting element in place looped about the soft tissue with both end portions extending from the same location of the body.

30. The routing method of claim 24, further providing a flexible guiding wire capable of engaging the flexible cutting element and maintaining engagement therewith under a tensile load, and wherein the routing tool has an internal lumen for receiving the wire, and further comprising:
extending the routing tool into the body at a first location adjacent to the soft tissue and out of the body at a second location adjacent of the tissue and opposite the first location so as to traverse the tissue on a first side;
extending the wire through the routing tool to leave a middle portion of the wire received within the routing tool;
re-tracking the tool from the body so as to leave the wire in place; extending the routing tool into the body at the first location and out of the body at a second location so as to traverse the tissue on a second side and opposite the first side;
extending the wire from the distal end to the proximal end of the routing tool while keeping the in-body portion of the wire in place;
re-tracking the routing tool from the body so as to hereby leave the wire in place looped about the soft tissue with both end portions extending from the same location of the body; and
engaging the flexible cutting element into the body to take the place of the wire.

31. The routing method of claim 18, wherein the routing tool is a hypodermic needle, and wherein the steps further comprise:
injecting liquid into the body to hydraulically clear up the routing access about the soft tissue.

32. The routing method of claim 18, wherein the routing tool comprises a first needle with an internal lumen and a solid needle capable of facilitating insertion into and extension through the internal lumen of the first needle to guide the direction of the tool.

33. The routing method of claim 32, wherein said solid needle is elastic or flexible and has a tip configured to direct at a designed angle under the contact forces during routing.

34. The routing method of claim 32, wherein said solid needle is made of Nitinol alloy or other shape-memory alloys, and pre-memory-shaped with a curved tip portion, and kept in a straight shape below a transition temperature assigned to be a body temperature;
extending the first needle into the body; and
inserting solid needle into the first needle, and further extend out of the first needle for designed length, at the body temperature the solid needle returns the original curve shape at the tip portion so as to guide the tool in designed direction.

35. A transecting system for transecting a soft tissue in a body, comprising:
a flexible cutting element, and
a routing tool for routing the flexible cutting element about the soft tissue in the body; and
an imaging device capable of visualizing the interior of the body.

36. The transecting system of claim 35, wherein the imaging device comprises an ultrasound imaging device.

37. The transecting system of claim 35, wherein said soft tissue is a ligament or tendon.

38. The transecting system of claim 35, wherein said target soft tissue is the transverse carpal ligament or the annular ligament of fingers or the laciniate ligament of foot or the transverse intermetatarsal ligament of foot or the aponeurosis of foot or the plantar fascia of foot or Achilles tendon of foot.

39. The transecting system of claim 35, wherein the routing tool is configured to be capable of routing the flexible cutting element about the soft tissue without the need to search, match, connect, engage or assemble the routing tool relative to the flexible cutting element or one component of the routing tool relative to another component, at a location inside of the body.

40. The transecting system of claim 35, wherein the routing tool comprises a unitary component.

41. The transecting system of claim 35, wherein the routing tool is a needle-like member having a length sufficient to extend from a first location adjacent to the soft tissue to be transected to a second location transversely adjacent to the soft tissue to be transected.

42. The transecting system of claim 35, wherein the routing tool has a configuration for engaging the cutting element and maintaining engagement therewith under a tensile load.

43. The transecting system of claim 42, wherein the configuration of the routing tool includes a hook-shaped void.

44. The transecting system of claim 42, wherein the configuration of the routing tool includes an eye-shape void.

45. The transecting system of claim 35, wherein the routing tool has an internal lumen for receiving the flexible cutting element and the flexible cutting element has at least one stiffened end section to facilitate insertion into and extension through the internal lumen of the routing tool.

46. The transecting system of claim 35, further comprising a flexible guiding wire capable of engaging the flexible cutting element and maintaining engagement therewith under a tensile load, and wherein the routing tool has an internal lumen for receiving the wire.

47. The transecting system of claim 35, wherein the routing tool is a hypodermic needle capable of injecting liquid into the body to hydraulically clear up the routing access about the soft tissue.

48. The transecting system of claim 35, wherein the routing system comprises a first needle with an internal lumen and a solid needle capable of facilitating insertion into and extension through the internal lumen of the first needle to guide the direction of both needles.

49. The transecting system of claim 48, wherein said solid needle is elastic or flexible and has a tip configured to direct at a designed angle during routing.

50. The transecting system of claim 48, wherein said solid needle is made of Nitinol Alloy or other shape-memory alloys, and pre-memory-shaped with a curved tip portion, and kept in a straight shape below a transition temperature assigned to be a body temperature; and capable of being inserted into the first needle, and further extension out of the first needle for designed length, and returning the original curved shape at the tip portion for guiding both needles in designed direction.

51. The transecting system of claim 35, wherein the flexible cutting element is a thread.

52. The transecting system of claim 35, wherein the flexible cutting element is a wire.

53. The transecting system of claim 35, wherein the flexible cutting element has zero bend radius.

54. The transecting system of claim 35, wherein the flexible cutting element has a substantially smooth surface with uniform surface hardness.

55. The transecting system of claim 35, wherein the flexible cutting element has a soft surface such that there is no abrasion-effect during sliding on the targeted tissue or non-targeted tissue.

56. The transecting system of claim 35, wherein the flexible cutting element has uniform physical properties lengthwise.

57. The transecting system of claim 35, wherein the flexible cutting element has uniform cross-sectional geometry and dimensions lengthwise.

58. The transecting system of claim 35, wherein the flexible cutting element is only in one uniform piece lengthwise.

59. The transecting system of claim 35, wherein the flexible cutting element is configured to be capable of transecting the targeted soft tissue without the need to place a protecting or guiding cover or tube inside the body.

60. The transecting system of claim 35, further comprising a power tool or a manual tool for exerting force on the flexible cutting element to transect the soft tissue in the body.

61. The transecting system of claim 35, further comprising an isolative system for encasing a power tool and allowing the power tool to be used without sterilization.

62. The transecting system of claim 35, further comprising an isolative system for encasing a manual tool and allowing the manual tool to be used without sterilization.

63. A power tool for exerting force on a thread-like or wire-like cutting element to transect a tissue which is routed about by the cutting element in a body, comprising:
a structure including a handle portion for grasping the structure;
a drive motor housed within the structure;
a switch for actuating and controlling the drive motor; and
an actuating mechanism coupled to the drive motor, and for providing alternating releasing-pulling actions, the alternating releasing-pulling actions being transferred through output portions to both ends of the cutting element separately and being capable of moving the cutting element for transecting the tissue.

64. The power tool of claim 63, wherein the actuating mechanism moves the cutting element alternately in opposite directions in a designed distance and at a designed frequency.

65. The power tool of claim 64, wherein the designed distant is in the range of 1 mm to 250 mm and designed frequency is in the range of 10 to 10000 times per minutes.

66. The power tool of claim 64, wherein the designed distance and the designed frequency are variable and controllable in use.

67. The power tool of claim 63, wherein the actuating mechanism is a crankshaft which includes:
a first crankpin; and
a second crankpin positioned in a 180° crank angle to the first crankpin, each crankpin being linked with one end of the flexible cutting element to exert releasing-pulling actions for transecting the tissue.

68. The power tool of claim 63, wherein the actuating mechanism includes:
a cylinder having a track formed on the cylinder, the cylinder being coupled to and rotatable by the drive means;
a first track follower; and
a second track follower, the first and second track followers being separately placed within the track of the cylinder and being movable in a reciprocating manner as the cylinder rotates.

69. The power tool of claim 63, wherein the actuating mechanism includes:
a first cam;
a first follower in contact with the first cam and movable in a reciprocating manner;
a second cam; and
a second follower in contact with the second cam and movable in a reciprocating manner in opposite direction of the first follower.

70. The power tool of claim 63, wherein the actuating mechanism includes:
a crankpin;
a crank connecting rod having a first end and a second end, the first end of crank connecting rod being engaged with the crankpin; and
a rocking arm engaged with the second end of crank connecting rod and movable in a rocking manner.

71. The power tool of claim 63, wherein the actuating mechanism includes:
a cam; and
a rocking arm with a follower having a fork-like structure on one end, the rocking arm being in contact with the cam by the follower and movable in a rocking manner.

72. The power tool of claim 63, further comprising a flexible guiding tube dimensioned to receive the flexible cutting element.

73. The power tool of claim 63, wherein the drive motor rotates a shaft that is coupled to the actuating mechanism.

74. The power tool of claim 63, further including a power source for powering the drive motor.

75. An isolative system for allowing a power tool to be used without the need for sterilization, capable of encasing the power tool having a structure including a handle portion and a mechanism for providing alternative releasing-pulling actions to a thread-like or wire-like cutting element by two output portions, and for transferring the alternative releasing-pulling actions of the power tool outside of the bag, the isolative system comprising:
a thin wall bag made from a flexible and soft material which is able to be sterilized and impermeable to germs and/or bacteria;
an opening in the bag for receiving the power tool;
a locking component associated with the opening for sealing the opening; and
a mechanism of transferring the alternative releasing-pulling actions of the power tool to the outside of the bag and to the cutting element.

76. The isolative system of claim 75, wherein the wall of the bag is visually penetrable.

77. The isolative system of claim 75, wherein the mechanism for transferring the alternating releasing-pulling actions comprises two holders configured to be separately held on the output portions of the power tool from the outside of the bag and being capable of transferring the alternative releasing-pulling actions of the power tool from inside of the bag to the outside the bag and to the cutting element.

78. The isolative system of claim 77, wherein the holders are cable tie type components.

79. The isolative system of claim 77, wherein the holders are locking ring or spring ring type components.

80. The isolative system of claim 77, wherein the holders are hook type components.

81. The isolative system of claim 77, wherein the holders are knots of the cutting element tied on the power tool from the outside of the bag.

82. The isolative system of claim 75, the mechanism for transferring the alternating releasing-pulling actions comprises a pair of relatively stiff housings attached and sealed to the surface of main body of the bag, each housing being capable of coupling with one of the output portions of the power tool for transferring the alternative releasing-pulling actions of the power tool from inside to outside of the bag and further transferring the alternative releasing-pulling actions to the cutting element.

83. The isolative system of claim 75, wherein the mechanism for transferring the alternating releasing-pulling actions further comprises a pair of locking components in the housings for keeping each housing coupling with an output portion of power tool.

84. The isolative system of claim 75, wherein the locking component is adhesive tape, rubber band or sealable lock.

85. A method for transecting a targeted soft tissue within a body, comprising the steps of:
providing a flexible cutting element;
providing a routing tool;
routing the flexible cutting element about the soft tissue utilizing the routing tool such that both ends of the flexible cutting element extend from a single access port in the body;
providing an imaging device and imaging said routing tool relative to said soft tissue as said tool is used to rout said cutting element about said tissue; and
exerting forces on the ends of the flexible cutting element to transect the soft tissue.

86. The method of claim 85, wherein the imaging device comprises an ultrasound imaging device.

87. The method of claim 85, wherein said targeted soft tissue is a ligament or tendon.

88. The method of claim 85, wherein said target soft tissue is the transverse carpal ligament or the annular ligament of fingers or the laciniate ligament of foot or the transverse intermetatarsal ligament of foot or the aponeurosis of foot or the plantar fascia of foot or Achilles tendon of foot.

89. The method of claim 85, wherein the exerting forces generate alternative releasing-pulling actions on the ends of the flexible cutting element to transect the soft tissue.

90. The method of claim 89, further including:
attaching a powered hand tool to the ends of the flexible cutting element for providing the alternative releasing-pulling actions to the flexible cutting element to transect the soft tissue.

91. The method of claim 89, further including:
attaching a manual tool to the ends of the flexible cutting element for providing the alternative releasing-pulling actions to the flexible cutting element to transect the soft tissue.

92. The method of claim 90, further including:
placing the powered hand tool into an isolative system which encases the power tool and is capable of transferring the power from inside the isolative system to outside the isolative system and further to the ends of the cutting element so as to allow the power tool to be used without sterilization.

93. The method of claim 91, further including:
placing the manual tool into an isolative system which encases the manual tool and is capable of operating hand turning crank of the manual tool from outside the isolative system and transferring the power from inside the isolative system to outside the isolative system and further to the ends of the cutting element so as to allow the manual tool to be used without sterilization.

94. The method of claim 92, wherein the isolative system is made from a thin wall bag made from a flexible and soft material which is able of being sterilized and is impermeable to germs and/or bacteria.

95. The method of claim 93, wherein the isolative system is made from a thin wall bag made from a flexible and soft material which is able of being sterilized and is impermeable to germs and/or bacteria.

96. The method of claim 85, wherein the flexible cutting element is a thread.

97. The method of claim 85, wherein the flexible cutting element is a wire.

98. The method of claim 85, wherein the flexible cutting element has zero bend radius.

99. The method of claim 85, wherein the flexible cutting element has a substantially smooth surface with uniform surface hardness.

100. The method of claim 85, wherein the flexible cutting element has a soft surface such that there is no abrasion-effect on the targeted tissue or non-targeted tissue during the sliding of the cutting element.

101. The method of claim 85, wherein the flexible cutting element has uniform physical properties lengthwise.

102. The method of claim 85, wherein the flexible cutting element has uniform cross-sectional geometry and dimensions lengthwise.

103. The method of claim 85, wherein the flexible cutting element is in one uniform piece lengthwise.

104. The method of claim 85, wherein the flexible cutting element is capable of transecting said targeted soft tissue without the need to place a protecting or guiding cover or tube inside the body.

105. The method of claim 85, wherein the routing tool comprises a first needle with an internal lumen and a solid needle capable of facilitating insertion into and extension through the internal lumen of the first needle to guide the direction of the tool.

106. The method of claim 105, wherein said solid needle is elastic or flexible and has a tip configured to direct at a designed angle under the contact forces during routing.

107. The method of claim 105, wherein said solid needle is made of Nitinol alloy or other shape-memory alloys, and pre-memory-shaped with a curved tip portion and kept in a straight shape below a transition temperature assigned to be a body temperature, and further comprising:
extending the first needle into the body; and
inserting the solid needle into the first needle, and further extending out the first needle for a designed length, such that at body temperature the solid needle returns the original curved shape at the tip portion so as to guide the tool in designed direction.

108. A manual tool for exerting force on a thread-like or wire-like cutting element to transect a tissue which is routed about by the cutting element in a body, comprising:
a structure including a handle portion for grasping the structure;
a hand turning crank mechanism; and
an actuating mechanism coupled to the hand turning crank mechanism, and for providing alternating releasing-pulling actions, the alternating releasing-pulling actions being transferred through output portions to both ends of the cutting element separately and being capable of moving the cutting element for transecting the tissue.

109. The manual tool of claim 108, wherein the actuating mechanism moves the cutting element alternately in opposite directions in a designed distance and at a designed frequency.

110. The manual tool of claim 109, wherein the designed distant is in the range of 1 mm to 250 mm and designed frequency is in the range of 10 to 10000 times per minutes.

111. The manual tool of claim 109, wherein the designed distance and the designed frequency are variable and controllable in use.

112. The manual tool of claim 108, wherein the actuating mechanism is a crankshaft which includes:
a first crankpin; and
a second crankpin positioned in a 180° crank angle to the first crankpin, each crankpin being linked with one end of the flexible cutting element to exert releasing-pulling actions for transecting the tissue.

113. The manual tool of claim 108, wherein the actuating mechanism includes:
a cylinder having a track formed on the cylinder, the cylinder being coupled to and rotatable by the drive means;
a first track follower; and
a second track follower, the first and second track followers being separately placed within the track of the cylinder and being movable in a reciprocating manner as the cylinder rotates.

114. The manual tool of claim 108, wherein the actuating mechanism includes:
a first cam;
a first follower in contact with the first cam and movable in a reciprocating manner;
a second cam; and
a second follower in contact with the second cam and movable in a reciprocating manner in opposite direction of the first follower.

115. The manual tool of claim 108, wherein the actuating mechanism includes:
a crankpin;
a crank connecting rod having a first end and a second end, the first end of crank connecting rod being engaged with the crankpin; and
a rocking arm engaged with the second end of crank connecting rod and movable in a rocking manner.

116. The manual tool of claim 108, wherein the actuating mechanism includes:
a cam; and
a rocking arm with a follower having a fork-like structure on one end, the rocking arm being in contact with the cam by the follower and movable in a rocking manner.

117. The manual tool of claim 108, further comprising a flexible guiding tube dimensioned to receive the flexible cutting element.

118. An isolative system for allowing a manual tool to be used without the need for sterilization, capable of encasing the manual tool having a structure including a handle portion, a mechanism of hand turning crank and a mechanism for providing alternative releasing-pulling actions to a thread-like or wire-like cutting element by two output portions, and for transferring the alternative releasing-pulling actions of the manual tool outside of the bag, the isolative system comprising:
a thin wall bag made from a flexible and soft material which is able to be sterilized and impermeable to germs and/or bacteria and big enough to allow the operation of hand turning crank from outside of the bag;
an opening in the bag for receiving the manual tool;
a locking component associated with the opening for sealing the opening; and
a mechanism of transferring the alternative releasing-pulling actions of the manual tool to the outside of the bag and to the cutting element.

119. The isolative system of claim 118, wherein the wall of the bag is visually penetrable.

120. The isolative system of claim 118, wherein the mechanism of transferring the alternative releasing-pulling actions comprises two holders configured to be separately held on the output portions of the tool from the outside of the bag and being capable of transferring the alternative releasing-pulling actions of the tool from inside of the bag to the outside the bag and to the cutting element.

121. The isolative system of claim 120, wherein the holders are cable tie type components.

122. The isolative system of claim 120, wherein the holders are locking ring or spring ring type components.

123. The isolative system of claim 120, wherein the holders are hook type components.

124. The isolative system of claim 120, wherein the holders are knots of the cutting element tied on the power tool from the outside of the bag.

125. The isolative system of claim 118, wherein the mechanism of transferring the alternative releasing-pulling actions comprises a pair of relatively stiff housings attached and sealed to the surface of main body of the bag, each housing being capable of coupling with one of the output portions of the tool for transferring the alternative releasing-pulling actions of the tool from inside to outside of the bag and further transferring the alternative releasing-pulling actions to the cutting element.

126. The isolative system of claim 118, wherein the mechanism of transferring the alternative releasing-pulling actions further comprises a pair of locking components in the housings for keeping each housing coupling with an output portion of said tool.

127. The isolative system of claim 118, wherein the locking component is adhesive tape, rubber band or sealable lock.
